(19) United States
(12) Patent Application Publication
(10) Pub. No.: US 2026/0369422 A1
(43) Pub. Date: Nov. 19, 2026

(54) SYSTEMS AND METHODS FOR HEALTHCARE RISK MANAGEMENT SYSTEM AND MULTI-DIMENSIONAL RISK INDEX

(71) Applicant: RLDATIX NORTH AMERICA INC., Toronto (CA)

(72) Inventors: Yves CREHORE, Toronto (CA); Mike SMITH, Toronto (CA)

(21) Appl. No.: 19/675,967

(22) Filed: May 13, 2026

Related U.S. Application Data
(60) Provisional application No. 63/805,036, filed on May 13, 2025.

(51) Int. Cl. G16H 50/30; G16H 40/67

(52) U.S. Cl. CPC G16H 50/30; G16H 40/67

(57) ABSTRACT

According to an aspect, there is provided systems and methods for healthcare risk management system. Non-transitory memory stores one or more risk models and configurations for model dimensions and weights. A processing system of one or more hardware processors is configured to receive the configurations for model dimensions and weights, and update the configurations stored in the memory; receive input data from a plurality of input data streams, and standardize the input data; compute a multidimensional risk index for healthcare event; and initiate an action, intervention, or event based on the multidimensional risk index.

### CROSS-REFERENCE

This application is a non-provisional of, and claims all benefit, including priority to, U.S. Provisional Patent Application No. 63/805,036, entitled “SYSTEMS AND METHODS FOR HEALTHCARE RISK MANAGEMENT SYSTEM AND MULTI-DIMENSIONAL RISK INDEX”, which is hereby incorporated by reference in its entirety.

### FIELD

Embodiments described herein relate to the field of selective visual display systems, computing systems, distributed systems, and healthcare. Embodiments relate to devices, systems, methods and non-transitory computer-readable storage mediums for healthcare risk management systems.

### INTRODUCTION

Embodiments described herein relate to computer systems for healthcare risk management. Embodiments described herein relate to computer systems that process different data streams, automatically generate multi-dimensional risk measurements, and trigger corresponding actions and alerts. Embodiments described herein relate to computer systems that integrate different data sources for multi-dimensional data risk analytics to improve healthcare systems.

There exists a need for computer systems, devices, systems, architectures, methods of operation, user interaction, and non-transitory computer-readable storage mediums for capturing and computing risk measurements for use with different healthcare systems, or at least alternatives. There exists a need for devices, systems, methods and non-transitory computer-readable storage mediums that integrate different data sources for multi-dimensional data risk analytics to proactively detect, prioritize, and mitigate risks in real-time to improve patient safety and operational resilience in healthcare systems. There exists a need for improved tools and technology, such as devices, systems, methods, and software-as-a-service applications, that can capture and compute risk measurements, or at least alternatives.

### SUMMARY

Described herein are systems, methods, cloud-based computing environments, and/or non-transitory computer-readable storage mediums for healthcare risk management systems.

Embodiments relate to devices, systems, methods, and non-transitory computer-readable storage mediums for healthcare risk management systems incorporating integrated scoring systems including a Harmonized Harm Score (HHS), a Multi-Dimensional Risk Index (MDRI), and Context-Based Risk Surveillance (CBRS).

Embodiments of the systems described herein may be a solution hosted in cloud-based storage devices that can be integrated with other applications. Embodiments of the systems described herein may be a solution hosted in on premise storage devices.

Embodiments relate to devices, systems, methods and non-transitory computer-readable storage mediums for capturing and computing risk measurements for use with different healthcare systems.

Embodiments relate to devices, systems, methods and non-transitory computer-readable storage mediums that integrate different data sources for multi-dimensional data risk analytics to proactively detect, prioritize, and mitigate risks in real-time to improve patient safety and operational resilience in healthcare systems.

In an aspect, embodiments described herein relate to a healthcare risk management system for providing visual elements for an interface. The system has: a processing subsystem comprising one or more processors and one or more memories coupled with the one or more processors, the processing subsystem configured to cause the system to: receive input data from a plurality of input data streams and standardize the input data; receive or determine, for a detected healthcare event, harmonized harm scores at least in part of the standardized input data; compute a current multidimensional risk index value for the healthcare event from the harmonized harm scores using a configurable risk model that defines a set of active dimensions, normalization values for each active dimension and weights for each active dimension, the current multidimensional risk index value comprising a total weighted normalized score across the active dimensions; fit a smoothing model to a historical time series of multidimensional risk index values and derive an expected risk trajectory with dynamically computed control bands; compare the current multidimensional risk index value against the expected risk trajectory and the dynamically computed control bands, and, upon an exceedance, initiate an action or intervention based on the multidimensional risk index value; generate visual elements corresponding at least in part to a portion of the multidimensional risk index value and the action or intervention initiated; and transmit the visual elements to an interface of a client device for display.

In some embodiments, the smoothing model is a regression-based model having the form y=β0+β1x+β2x²+ . . . +βnxⁿ+ε, where y is an expected multidimensional risk index value for a reporting period x, β0 is an intercept, β1 through βn are coefficients fitted from the time series of multidimensional risk index values, n is a polynomial degree, and ε is a residual variation.

In some embodiments, the smoothing model is selectable from a plurality of methods comprising a polynomial regression, a rolling average, a moving average, and an exponentially weighted moving average.

In some embodiments, the dynamically computed control bands are produced, from a standard deviation of the expected risk trajectory, at ±1 standard deviation and ±2 standard deviations from the expected risk trajectory.

In some embodiments, configurations for the configurable risk model indicate specific dimensions representing risk categories, normalization min scale values and max scale values for each dimension, and weights for each dimension.

In some embodiments, the system has the client device with the interface to receive updated configurations for the configurable risk model and display at least a portion of the visual elements generated at least in part corresponding to a portion of the multidimensional risk index, and wherein the processing subsystem is further configured to receive the updated configurations for the configurable risk model and recompute the current multidimensional risk index value for the healthcare event from the harmonized harm scores and generate updated visual elements for display at the interface.

In some embodiments, the processing subsystem is further configured to stratify a current multidimensional risk index value, by reference to the expected risk trajectory and the dynamically computed control bands, into one of: a nominal state, a watch state corresponding to a +1 standard deviation exceedance, a sentinel state corresponding to a +2 standard deviation exceedance, and a drift state corresponding to a sustained directional deviation from the expected risk trajectory over a configurable number of reporting periods, and select a structured intervention work-item in dependence upon the stratified state.

In some embodiments, the processing subsystem is further configured to ingest harmonized harm scores from, and route the action or intervention to, at least one of: a nurse call system, a medication-dispensing cabinet, a patient-monitoring device, and a scheduling system to initiate a downstream control action.

In some embodiments, the processing subsystem integrates scoring systems comprising one or more of: Harmonized Harm Score (HHS), a Multi-Dimensional Risk Index (MDRI), and Context-Based Risk Surveillance (CBRS).

In some embodiments, the processing subsystem further includes role-based access controls to provide access for risk model configuration, MDRI analysis, and intervention management to authorized users based on predefined access roles and organizational hierarchies.

In an aspect, embodiments described herein relate to a method for healthcare risk management. The method involves: receiving input data from a plurality of input data streams and standardizing the input data; receiving or determining, for a detected healthcare event, harmonized harm scores at least in part from the standardized input data; computing a current multidimensional risk index value for the healthcare event from the harmonized harm scores using a configurable risk model that defines a set of active dimensions, normalization values for each active dimension and weights for each active dimension, the current multidimensional risk index value comprising total weighted normalized score across active dimensions; fitting a smoothing model to a historical time series of multidimensional risk index values and derive an expected risk trajectory with dynamically computed control bands; comparing the current multidimensional risk index value against the expected risk trajectory and the dynamically computed control bands, and, upon an exceedance, initiating an action or intervention based on the multidimensional risk index value; generating, for display at a visual interface, visual elements corresponding at least in part to a portion of the multidimensional risk index value and the action or intervention initiated; and transmitting the visual elements to an interface of a client device.

In some embodiments, the smoothing model is a regression-based model having the form y=β0+β1x+β2x²+ . . . +βnxⁿ+ε, where y is an expected multidimensional risk index value for a reporting period x, β0 is an intercept, β1 through βn are coefficients fitted from the time series of multidimensional risk index values, n is a polynomial degree, and ε is a residual variation.

In some embodiments, the smoothing model is selectable from a plurality of methods comprising a polynomial regression, a rolling average, a moving average, and an exponentially weighted moving average.

In some embodiments, the dynamically computed control bands are produced, from a standard deviation of the expected risk trajectory, at ±1 standard deviation and ±2 standard deviations from the expected risk trajectory.

In some embodiments, the configurations for the configurable risk model indicate specific dimensions representing risk categories, normalization min scale values and max scale values for each dimension, and weights for each dimension.

In some embodiments, the method involves receiving updated configurations for the configurable risk model and recomputing the current multidimensional risk index value for the healthcare event from the harmonized harm scores.

In some embodiments, the method involves stratifying a current multidimensional risk index value, by reference to the expected risk trajectory and the dynamically computed control bands, into one of: a nominal state, a watch state corresponding to a +1 standard deviation exceedance, a sentinel state corresponding to a +2 standard deviation exceedance, and a drift state corresponding to a sustained directional deviation from the expected risk trajectory over a configurable number of reporting periods, and selecting a structured intervention work-item in dependence upon the stratified state.

In some embodiments, the method involves ingesting harmonized harm scores from, and routing the action or intervention to, at least one of: a nurse call system, a medication-dispensing cabinet, a patient-monitoring device, and a scheduling system to initiate a downstream control action.

In some embodiments, the method involves controlling access for risk model configuration, MDRI analysis, and intervention management to authorized users based on predefined access roles and organizational hierarchies.

In an aspect, embodiments described herein relate to non-transitory computer-readable medium to store instructions, which when executed by a computer, cause the computer to perform operations comprising: receiving input data from a plurality of input data streams and standardizing the input data; receiving or determining, for a detected healthcare event, harmonized harm scores at least in part based on the standardized input data; computing a current multidimensional risk index value for the healthcare event from the harmonized harm scores using a configurable risk model, the current multidimensional risk index value comprising total weighted normalized score across active dimensions; fitting a smoothing model to a historical time series of multidimensional risk index values and deriving an expected risk trajectory with dynamically computed control bands; comparing the current multidimensional risk index value against the expected risk trajectory and the dynamically computed control bands, and, upon an exceedance, initiating an action or intervention based on the multidimensional risk index value; generating, for display at a visual interface, visual elements corresponding at least in part to a portion of the multidimensional risk index value and the action or intervention initiated; and transmitting the visual elements to an interface of a client device.

In an aspect, embodiments described herein provide for integration and interoperability of the scoring systems (HHS, MDRI, CBRS). In an aspect, embodiments described herein provide for customizable weighting and normalization tools. In an aspect, embodiments described herein provide for real-time analytics and evidenced and defined interventions enabled by the system.

In an aspect, embodiments described herein provide a healthcare risk management system for providing visual elements for an interface. The system involves non-transitory memory storing one or more risk models and configurations for model dimensions and weights; a processing system of one or more hardware processors programmed with executable instructions and with a communication path to the non-transitory memory, wherein the processing system is configured to: receive the configurations for model dimensions and weights, and update the configurations stored in the memory; receive input data from a plurality of input data streams, and standardize the input data; detect a healthcare event; compute a multidimensional risk index for the event; and initiate an action or intervention based on the multidimensional risk index. The system involves a computer device with a hardware processor having a client application with an interface to receive the configurations for model dimensions and weights and display visual elements generated at least in part corresponding to a portion of the multidimensional risk index, and the action.

In some embodiments, the processing system is configured to generate an appropriate treatment or measure for mitigating one or more risks based on the multidimensional risk index and the action, and generate one or more visual elements for the appropriate treatment or measure for display at the interface.

In some embodiments, the processing system receives updated configurations for the model dimensions and weights, recomputes the multidimensional risk index for the event, and initiates a different action based on the recomputed multidimensional risk index.

In some embodiments, the configurations for model dimensions and weights are for different organizations, wherein a configuration for model dimensions and weights is associated with an identifier for the organization.

In some embodiments, the configurations for model dimensions and weights indicate specific dimensions used for the risk model, normalization min scale values and max scale values for each dimension, and weights for each dimension.

In some embodiments, the processing system computes the multidimensional risk index for the event by aggregating a total weighted score and a harmonized harm score.

In some embodiments, the total weighted score comprises a weighted normalized score for each dimension of the risk model.

In some embodiments, the processing system integrates scoring systems comprising Harmonized Harm Score (HHS), a Multi-Dimensional Risk Index (MDRI), and Context-Based Risk Surveillance (CBRS), generates the visual elements based on the scoring systems comprising HHS, MDRI and CBRS, and transmits the visual elements to the interface for display.

In some embodiments, the processing system is configured for capturing and computing risk measurements for use with different healthcare systems and generates visual elements specific to the different healthcare systems, the visual elements indicating at least a portion of the risk measurements.

In some embodiments, the processing system is configured to integrate different data sources for multi-dimensional data risk analytics to proactively detect, prioritize, and mitigate risks in real-time to improve patient safety and operational resilience in healthcare systems, and generate the visual elements comprising one or more indicators for proactively detect, prioritize, and mitigate risks.

In some embodiments, the processing system is configured for integration and interoperability of the scoring systems comprising HHS, MDRI, CBRS, and generates customizable weighting and normalization tools.

In some embodiments, the processing system is configured to generate the visual elements with indications of real-time analytics and evidenced and defined interventions.

In some embodiments, the processing system is configured as a multi-tenant software-as-a-service platform enabling each healthcare system of the different healthcare systems to securely access, configure, and analyze risk data without cross-access to other healthcare systems.

In some embodiments, the processing system further includes role-based access controls to provide access for dimensional score configuration, MDRI analysis and intervention management to only authorized users based on predefined access roles and organizational hierarchies.

In some embodiments, the processing system further comprises one or more application programming interfaces to permit the different data sources to send and receive data for MDRI computation.

In some embodiments, the processing system applies encryption-in-transit and encryption-at-rest protocols as security and compliance controls.

In some embodiments, the processing system further employs elastic compute resource scaling to dynamically adjust processing and storage resources based on incident volume and active user demand across the different healthcare systems.

In an aspect, embodiments described herein provide a method for healthcare risk management. The method involves: storing one or more risk models and configurations for model dimensions and weights; receiving the configurations for model dimensions and weights, and updating the configurations stored in the memory; detecting a healthcare event; receiving input data from a plurality of input data streams, and populating one or more dimensions of a risk model with standardized input data; computing a multidimensional risk index for the event; initiating an action based on the multidimensional risk index; and generating visual elements at least in part corresponding to a portion of the multidimensional risk index, and the action.

In an aspect, embodiments described herein provide non-transitory computer-readable medium to store instructions, which when executed by a computer, cause the computer to perform operations comprising: receiving the configurations for model dimensions and weights, and updating configurations stored in the memory; receiving input data from a plurality of input data streams, and populating one or more dimensions of a risk model with standardized input data; computing a multidimensional risk index for a healthcare event; initiating an action based on the multidimensional risk index; and generating visual elements at least in part corresponding to a portion of the multidimensional risk index, and the action.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 shows an example system for healthcare risk management, according to some embodiments;

FIG. 2A is a process flow for healthcare risk management, according to some embodiments;

FIG. 2B is a process flow for healthcare risk management and context-based risk surveillance, according to some embodiments;

FIG. 3 is a schematic of data flow for healthcare risk management using Harmonized Harm Score (HHS), Multi-Dimensional Risk Index (MDRI) and Context-Based Risk Surveillance (CBRS) systems, according to some embodiments;

FIG. 4 is a schematic of data flow for applications relating to healthcare risk management, according to some embodiments;

FIG. 5 is a visualization of harm score levels for categorizing incidents based on harm caused or the potential for harm, according to some embodiments;

FIG. 6 shows a stacked bar chart visualization of example incidents categorized based on risk severity on the HHS scale, according to some embodiments;

FIG. 7 shows a pie chart visualization of example events or incidents categorized based on risk severity on the HHS scale, according to some embodiments;

FIG. 8 shows a line graph visualization of example incidents over time categorized based on risk severity on the HHS scale, according to some embodiments;

FIG. 9 shows an example table of weighted risk scores, according to some embodiments;

FIG. 10A-10B show tables of weighted risk scores with different weightings for each dimension of risk, according to some embodiments;

FIG. 11 shows an example user interface dashboard for users to score events or incidents on multiple dimensions of risk, according to some embodiments;

FIG. 12 shows an example user interface dashboard for users to submit details of events or incidents, according to some embodiments;

FIG. 13 shows a stacked bar chart visualization of example weighted normalized scores of multiple risk dimensions, according to some embodiments;

FIG. 14 shows a pie chart visualization of an example MDRI count of events, according to some embodiments;

FIG. 15 shows a line graph visualization of an example average final MDRI score trend, according to some embodiments;

FIG. 16 shows an example Key Performance Indicator (KPI) style card, according to some embodiments;

FIG. 17 shows a line graph visualization of example incident rates obtained using Context-Based Risk Surveillance (CBRS) over time with highlighted significant events, according to some embodiments;

FIG. 18 shows a line graph visualization of example average MDRI scores over time with highlighted significant events, according to some embodiments;

FIG. 19 shows a heat map visualization of example MDRI scores per day over 31 days, according to some embodiments;

FIG. 20 shows a bar graph visualization of example average MDRI scores per day over 31 days, according to some embodiments;

FIG. 21 shows an example schematic for a system for healthcare risk management, according to some embodiments;

FIG. 22 shows an example schematic for a system for healthcare risk management with a visual interface, according to some embodiments; and

FIG. 23 is a schematic diagram of an example computing device, according to some embodiments.

### DETAILED DESCRIPTION

Described herein are systems and methods, devices, and non-transitory computer-readable storage mediums for healthcare risk management.

Traditional risk models may focus narrowly on patient harm, thereby missing broader organizational risks with financial, operational, and reputational impacts. Risk models may lack forecasting and multi-dimensional risk assessments, leading to missed critical intervention points and inefficient processes.

There is a lack of comprehensive risk profiling when it comes to events which happen in a healthcare system. Embodiments described herein can capture multiple dimensions of risk which have traditionally gone unreported, develop a configurable scoring system to calculate a more comprehensive risk score or measurement, identify dimensional concerns, and automatically generate and disseminate alerts based on the risk measurements. The reduction or prevention of event occurrence can be substantially maximized when multiple dimensions of risk are reviewed and can be acted on. For example, when all events are profiled, events with lower patient harm but high reputational risk can be uncovered whereas they may have otherwise gone unnoticed.

Root cause analysis (RCA) on events help to identify some of the causes for an event occurring. However, RCAs do not identify the impact that the event has had on other areas of the organization. Failure Mode and Effects Analyses (FMEAs) can identify potential risks and determine what their impact could be if the event were to occur, however these are conducted mostly on departmental or corporate risks (e.g. what if the cost of supplies increases by 10%). Embodiments described herein can use a bottom up approach for risk management, which puts the tracking of multiple dimensions of risk directly within the event, enabling the ability to intervene on risk both immediately and systemically. Embodiments described herein can focus on incidents with frequent recurrence to address systemic flaws.

As an example, for patient harm events (e.g. adverse event reports, incidents), organizations may only score the “severity” of the event based on patient harm. However, low harm or no harm events could actually result in major organizational harm which is going undetected. This is an illustrative example of events and the scoring system can be applied across a wide range of healthcare events. As another example, patient feedback events (e.g., complaints) could also have dimensions introduced that organizations could score on. As a further example, a failed credentialing event where a physician does not get their credentials renewed could also utilize this scoring system.

Embodiments described herein can generate (and continuously) update a Multi-Dimensional Risk Index (MDRI) to provide a way for organizations to be notified and alerted to these previously undetected risks in such a way that they can immediately intervene on those risks (to reduce the organizational harm while the patient is within the hospitals care) and also capture and manage these risks systemically. Interventions can be conducted at multiple levels of severity. For example, interventions can be conducted at two levels of severity. The first level is if the risk of just one dimension (e.g., reputation) is high but all other dimension risks are low, then the MDRI score would likely not be high or concerning and a dimension-specific intervention could be utilized. The second level is if the risk indicates that several dimensions are involved with a high MDRI score. In this case, the same dimensional interventions would likely be utilized but the coordination of a high MDRI score would require more than one-dimension subject matter expert to manage the issue or event.

Embodiments described herein provide a method to integrate a Harmonized Harm Score (HHS), the MDRI, and Context-Based Risk Surveillance (CBRS) into a unified, evidence-driven scoring system designed to enhance patient safety and proactively manage risk in healthcare facilities.

The HHS is a standardized score designed to objectively measure and categorize the severity of patient safety events across all types of patient harm incidents. The HHS addresses the problem of inconsistent capture of patient harm within other industry frameworks and schemas. For example, the Agency for Healthcare Research and Quality (AHRQ) scoring system uses an A-I scale which has been proved to be difficult for a front-line staff member to accurately select a severity from. The HHS provides a consistent and reliable way for staff members to capture the dimension of patient harm, which is key to not only initiating effective workflows and notifications in the system but is also important for accurate calculation of MDRI scores (as described below). The purpose of the HHS is to establish a robust and effective Harm Severity Index framework for healthcare event reporting within software application platforms. Embodiments described herein align with industry standards while enhancing usability, improving compliance in reporting forms, initiating timely and meaningful workflows. The Harm Severity Index provides a level 1-5 scale for categorizing incidents based on actual harm or potential for harm, enabling entities to prioritize risks for immediate and targeted intervention, standardize assessments to consistently evaluate the impact of patient safety incidents, and facilitate reporting and learning to proactively mitigate risks and prevent recurrence.

The MDRI is a comprehensive, unified score that combines the HHS with multiple risk dimensions. Examples of risk dimensions include likelihood of recurrence, operational impact, detection/preventability, regulatory/organizational impact, financial impact, staffing and workforce impact, clinical process impact, public perception or media impact, and the like. The MDRI provides a holistic, actionable assessment of patient safety incidents. The MDRI provides healthcare entities and organizations with customizable, multi-layered insights designed to prioritize risks, guide proactive interventions, and effectively manage patient outcomes in alignment with global healthcare standards.

CBRS refers to the continuous, systematic collection, analysis, and interpretation of patient safety and risk-related data specific to a healthcare entity and the population it serves. CBRS leverages the MDRI and real-time, context-driven analytics to proactively identify, prioritize, and intervene in high-risk patient safety scenarios. CBRS facilitates targeted interventions to reduce patient harm and also offers healthcare leaders improved insight into overall organizational risk health. CBRS represents a transformative approach, providing a new way for healthcare organizations to visualize, interpret, and proactively manage patient and organizational risk.

Embodiments described herein relate to hardware and/or software (including on-premise or cloud-based solutions) to capture input data for the dimensions of risk, compute the MDRI, and generate interventions. In addition to a scoring, embodiments described herein relate to processes for alerting, generating notifications, intervention management, and so on.

Embodiments described herein relate to a risk scoring system that is more comprehensive and utilizes additional dimensions to capture other risk factors such as: reputational risk, detection or prevention risks, recurrence risk, operational risk, staff impact risk, resourcing risk, financial risk, litigation risk, regulatory compliance risk and any other risk factor an organization may want to capture to drive decision-making and scoring of risk factors. Embodiments described herein integrate harm severity, using the HHS score, as a dimension in the scoring system.

In some embodiments, the MDRI consolidates risk dimension data within a cloud-based system and generates a comprehensive and unified score that is consistent across organizations. In some embodiments, MDRI incorporates additional risk dimensions to enable data-driven decision-making processes. In some embodiments, MDRI uses standardized cloud-based data to assess risks comprehensively and accurately and generate comprehensive and unified scores that are consistent across organizations. This allows organizations to understand the holistic risk of adverse events and also intervene on dimensional concerns.

The MDRI extends beyond the scoring of events and can be used in other health management components. For example, for Policy Management the use of MDRI scoring in FMEA may consider “If this policy was not followed, what might the risk be to the organization”. As another example, credentialing can use MDRI scoring to identify the impact of a failed credential or privilege to the organization. As a further example, Feedback Management can use MDRI scoring to identify the impact of a complaint, compliment, or grievance.

The Multi-Dimensional Risk Index (MDRI) has a wide aperture of risk, which may result in various advantages or benefits such as reduced litigation costs, enhanced compliance, and optimized usage of resources. Healthcare organizations can gain valuable insights to drive proactive decision-making, improving patient outcomes and operational resilience. This data-driven approach to risk mitigation through intervention results in corrective actions which moves healthcare organizations from reactive harm assessment to proactive risk mitigation.

Embodiments described herein relate to the capture and computation of a Harmonized Harm Score and an MDRI score, as well as a system for overall risk reduction and event prevention.

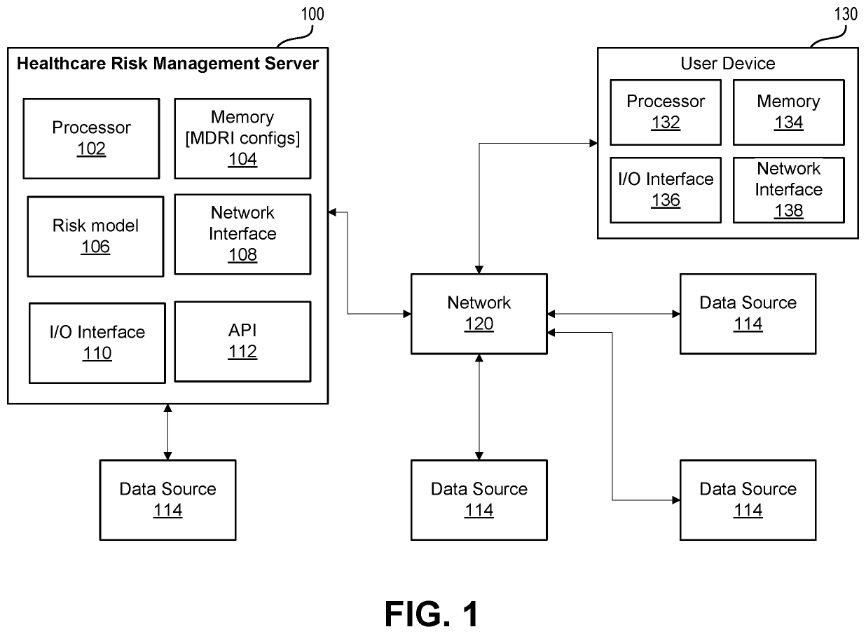

FIG. 1 shows an example system 100 for healthcare risk management according to some embodiments.

FIG. 1 illustrates an exemplary architecture for implementing operations of embodiments described herein. The architecture comprises a healthcare risk management server 100 (which may also be referred to as system 100 herein) that is configured to capture data from different data sources 114 and compute risk measurements for use with different healthcare systems, as well as other processes which will be described in greater detail herein. The system 100 is configured to communicate with network 120, user device 130 and data sources 114. The user device 130 and data sources 114 may include, for example, a Smartphone, a laptop, a computer, or other similar computing device. The user device 130 comprises a processor 132, a memory 134, an I/O interface 136 and a network interface 138. In some embodiments, user device 130 accesses system 100 via a web-browser residing thereon over a secure HTTPS connection which may be further secured using Transport Layer Security encryption for example. The user device 130 can connect out of their organization to the hosted system 100 where various components are embedded, and log in to an application to gain access to software components and files. In some embodiments, within an event file the user device 130 will then have access to MDRI user interface components provided by system 100. Data entered through the user interface components can be transmitted and stored in a database in memory 104, within a dedicated database table. In some embodiments, system 100 is a cloud-based or software as a service (SaaS) system. For risk specifically system 100 can have tenants or containers per client, and can follow a SaaS based deployment model. In some embodiments, dimensions and libraries of interventions can come from external data sources or additional client systems or other healthcare systems.

The system 100 may communicatively couple with the user device 130 and data sources 114 via a public or private network, such as network 120. Suitable networks may include or interface with any one or more of, for instance, a local intranet, a PAN (Personal Area Network), a LAN (Local Area Network), a WAN (Wide Area Network), a MAN (Metropolitan Area Network), a virtual private network (VPN), a storage area network (SAN), a frame relay connection, an Advanced Intelligent Network (AlN) connection, a synchronous optical network (SONET) connection, a digital T1, T3, E1 or E3 line, Digital Data Service (DDS) connection, DSL (Digital Subscriber Line) connection, an Ethernet connection, an ISDN (Integrated Services Digital Network) line, a dial-up port such as a V.90, V.34 or V.34bis analog modem connection, a cable modem, an ATM (Asynchronous Transfer Mode) connection, or an FDDI (Fiber Distributed Data Interface) or CDDI (Copper Distributed Data Interface) connection. Furthermore, communications may also include links to any of a variety of wireless networks, including WAP (Wireless Application Protocol), GPRS (General Packet Radio Service), GSM (Global System for Mobile Communication), CDMA (Code Division Multiple Access) or TDMA (Time Division Multiple Access), cellular phone networks, GPS (Global Positioning System), CDPD (cellular digital packet data), RIM (Research in Motion, Limited) duplex paging network, Bluetooth radio, or an IEEE 802.11-based radio frequency network. The network 120 can further include or interface with any one or more of an RS-232 serial connection, an IEEE-1394 (Firewire) connection, a Fiber Channel connection, an IrDA (infrared) port, a SCSI (Small Computer Systems Interface) connection, a USB (Universal Serial Bus) connection or other wired or wireless, digital or analog interface or connection, mesh or Digi® networking.

The system 100 comprises a processor 102, a memory 104, a risk model 106, a network interface 108, an I/O interface 110, and an application programming interface (API) 112, and a memory 140. According to some embodiments, the memory 104 comprises logic (e.g., instructions or applications) that can be executed by the processor 102 to perform various methods or operations. The risk model 106 and I/O interface 110 can be configured to capture input data from different data sources 114 for populating or updating the dimensions of risk, compute the MDRI, and generate interventions described herein. The risk model 106 can be a configurable risk model. In some embodiments, the risk model defines a set of active dimensions, normalization scales for each active dimension, and importance weights for each active dimension. System 100 can receive configurations for the model dimensions and update the configurable risk model. System 100 can also receive updated configurations to continue to update the configurable risk model.

Advantages of the systems and methods described herein include comprehensive risk management, resulting in reduced litigation costs, reduced revenue loss and optimized resource allocation. The systems and methods described herein further enhance regulatory compliance and minimize penalties by identifying systemic risks early.

Embodiments described herein prioritize interventions, mitigate systemic vulnerabilities, and enable proactive measures to prevent harm and enhance patient outcomes.

Embodiments described herein provide an integrated approach (using the HHS, MDRI, and CBRS) that enhances predictive accuracy, enabling more precise resource allocation, intervention planning, and regulatory compliance assurance.

Embodiments described herein further provide automated calculations and enhanced data interoperability to improve the ability of different systems, applications, and organizations to exchange, understand, and use data seamlessly. The proposed system's real-time analysis and alerting capabilities provide a comprehensive picture of risk to enable managerial intervention on risks that would otherwise be unknown or undetected, thus mitigating harm comprehensively and efficiently. The proposed system further leverages standardized scoring and data normalization across dimensions, independent of scoring scales and importance weightings, improving interoperability and enabling consistent benchmarking and cross-organizational comparisons.

Potential applications and use cases include usage of the systems and methods described herein by healthcare organizations to improve patient safety and enhance adherence to regulatory mandates, while strengthening the organizations' reputations in the industry. For example, the systems and methods described herein can be used for supply chain risk management to identify potential disruptions in hospital supply chains (events) and evaluating their risk impact to the organization across multiple dimensions, ensuring continuous operation and mitigating resource-related risks. As another example, embodiments described herein can be used for staff credentialing and privileging to proactively manage risk associated with staffing errors or inadequate training (events) and evaluating their risk impact to the organization across multiple dimensions, improving overall clinical governance.

Embodiments of the healthcare risk management server 100 (or system 100) are configured for providing visual elements for an interface (e.g. at user device 130). The system 100 has non-transitory memory 104 storing one or more risk models and configurations for model dimensions and weights. The system 100 has a processing system of one or more hardware processors 102 programmed with executable instructions and with a communication path to the non-transitory memory 104. The processing system is configured to: receive the configurations for model dimensions and weights, and update the configurations stored in the memory; receive input data from a plurality of input data streams, and standardize the input data; detect a healthcare event; compute a multidimensional risk index for the incident; and initiate an action, intervention, or event based on the multidimensional risk index.

User device 130 (or computer device) has a hardware processor 132 having a client application with an interface to receive the configurations for model dimensions and weights and display visual elements generated at least in part corresponding to a portion of the multidimensional risk index, and the event. The system 100 provides an improved display, and efficiently uses resources. For example, the signals representing visual elements may occupy significantly less bandwidth than the input data processed by system 100 to optimize use of network resources, given the reduced transmission and storage hardware required for the visual elements.

In some embodiments, the processing system transmits the visual elements to the interface of user device 130 over network 120 for display. The visual elements are generated by the processing system based on the computed multidimensional risk index and the initiated action or intervention, and are transmitted as structured display data (for example, as rendered graphical objects, dashboard components, or formatted data payloads) to the client application on user device 130. The client application receives the transmitted visual elements and renders them at the interface for viewing by an authorised user. The transmission operation can involve the processing system encoding the visual elements into a transmittable format, addresses the encoded data to the network interface 138 of user device 130, and dispatches the data over a communication path through network 120. In some embodiments, the processing system compresses or selectively filters the visual elements prior to transmission so that only the visual elements relevant to the user's role, organisation, and current context are transmitted, thereby reducing bandwidth consumption on network 120 and processing load on user device 130.

In some embodiments, the processing system is configured to generate an appropriate treatment or measure for mitigating one or more risks based on the multidimensional risk index and the event, and generate one or more visual elements for the appropriate treatment or measure for display at the interface user device 130.

In some embodiments, the processing system receives updated configurations for the model dimensions and weights, recomputes the multidimensional risk index for the incident, and initiates a different action based on the recomputed multidimensional risk index.

In some embodiments, the configurations for model dimensions and weights are for different organizations, wherein a configuration for model dimensions and weights is associated with an identifier for the organization.

In some embodiments, the configurations for model dimensions and weights indicate specific dimensions used for the risk model, normalization min scale values and max scale values for each dimension, and weights for each dimension.

In some embodiments, the processing system computes the multidimensional risk index for the incident by aggregating a total weighted score and a harmonized harm score.

In some embodiments, the total weighted score comprises a weighted normalized score for each dimension of the risk model.

In some embodiments, the system 100 integrates different scoring systems: Harmonized Harm Score (HHS), a Multi-Dimensional Risk Index (MDRI), and Context-Based Risk Surveillance (CBRS). The system 100 generates the visual elements based on the scoring systems comprising HHS, MDRI and CBRS, and transmits the visual elements to the interface at user device 130 for display.

In some embodiments, the processing system is configured for capturing and computing risk measurements for use with different healthcare systems, and generates visual elements specific to the different healthcare systems. The visual elements indicate at least a portion of the risk measurements.

In some embodiments, the processing system is configured to integrate different data sources for multi-dimensional data risk analytics to proactively detect, prioritize, and mitigate risks in real-time to improve patient safety and operational resilience in healthcare systems, and generate the visual elements comprising one or more indicators to proactively detect, prioritize, and mitigate risks.

In some embodiments, the processing system is configured for integration and interoperability of the scoring systems comprising HHS, MDRI, CBRS, and generates customizable weighting and normalization tools and transmits the tools to the user device 130 for display and usage.

In some embodiments, the processing system is configured to generate the visual elements with indications of real-time analytics and evidenced and defined interventions and transmits the visual elements to the interface at user device 130 for display.

In some embodiments, the processing system is configured as a multi-tenant software-as-a-service platform enabling each healthcare system of the different healthcare systems to securely access, configure, and analyze risk data without cross-access to other healthcare systems. This improves security and data protection, while sharing a common computational backend.

Each client or organization would need a tenant within the processing system to access where MDRI settings and data reside. The MDRI settings can be stored in a table of the database (where the dimensions, scales for each dimension, and importance weighting are defined), and data values would be stored in another table or series of related tables (where data related to the event ID from the reporting tool application would be stored along with dimension scores provided and any other relevant information that should be captured).

For example, system 100 can provide an interface with an HHS field to capture events. System 100 can define MDRI related settings and dimension data records. The dimension data records can define the dimension name, the dimension owners or entities, the dimension scale or scoring system, the importance weighting of the dimension, and so on. System 100 can also define alerts and conditions for the alerts. System 100 can provide a user interface to capture dimensional risk scores and data. System 100 can transmit dimensional values between components to calculate the MDRI for transmission and/or storage. System 100 can have internal alerting, notification, reporting, and dashboarding tools to use the MDRI and dimension data.

As another example, system 100 be a stand-alone tool for individual organizations. System 100 can allow each organization to identify and define dimensions data records, including dimension name, the dimension owners or entities, the dimension scale or scoring system, the importance weighting of the dimension, and so on. System 100 captures MDRI and CBRS related data, performs MDRI calculations, and initiates alerts and notifications.

In some embodiments, there may be UI elements in the system 100 that can be exposed to other applications (e.g. as part of a RESTful or GraphQL API), so that they embed needed form fields, inputs or settings from the system 100 into their application and/or event forms. If not embedding elements in their event forms, there could also just be an MDRI button in the event form that can open a UI in an application that is managed independently.

Users can define dimension values using a tool that can be embedded in system 100 or which reside in the MDRI tool. The dimension values can be sent to system 100 via an API (or an HTTPS request) to transmit needed data in various formats (e.g. JSON or XML format). The system 100 can implement an MDRI Calculation Engine or engage functions or services within a backend system to handle the MDRI computation. After the MDRI Calculation Engine processes the data, the calculated values can be returned to an external application via the same API that the request was sent through, such as via an HTTPS response, for example.

In some embodiments, each client may have a tenant within the system 100 where the MDRI settings and data will reside. Settings can be in a table of the database (e.g. where the dimensions are defined, scales for each dimension, importance weightings), and data values can be stored in another table, or series of related tables (e.g. where data related to the event ID from the system would be stored along with Dimension Scores Provided and any other relevant information captured).

In some embodiments, the processing system further includes role-based access controls (RBAC) to provide access for dimensional score configuration, MDRI analysis and intervention management to only authorized users based on predefined access roles and organizational hierarchies. This improves technical security. The RBAC enables authorized risk managers to configure scores and dimensions and involve only authorized subject matter experts. According to an aspect, RBAC can be implemented using a service-to-service approach, where a safelist of applications that are authorized to call API 112 is created. This implementation would be appropriate for users that just interact with UI elements to set dimension scores or if only data is being transmitted to server 100. According to another aspect, RBAC can be implemented with an embedded authentication system to allow users to log into the server 100. In some embodiments, there are three roles implemented in the RBAC: administrators who configure the MDRI settings, managers who access reporting tools/components within the server 100, and entry users who configure and control the RBAC.

In some embodiments, RBAC may be implemented as a SaaS solution, using one or more safelists of what applications can call the API. If users need to log into an MDRI tool, then there may be an authentication system built in. There can be different roles, such as an administrator role who configures the MDRI settings, a Manager role who has access to reporting tools or components within MDRI, and an Entry User role. For RBAC, there can be mechanisms in place to determine what roles can configure the MDRI settings, and what roles are able to interact with the data (such as entering in a dimension score).

In some embodiments, the processing system further comprises one or more application programming interfaces 112 to permit the different data sources 114 to send and receive data for MDRI computation. The APIs 112 can permit external data sources, including electronic medical records (EMRs), laboratory systems, and third-party reporting platforms, to send and receive data for MDRI computation and CBRS updates.

In some embodiments, the application programming interfaces 112 are deliberately bidirectional. The APIs 112 not only receive incident, dimensional, contextual, and configuration inputs from data sources 114 (including electronic medical records (EMRs), laboratory information systems, third-party reporting platforms, scheduling systems, and patient monitoring equipment), but also return, to the calling external system, a structured response object that includes one or more of: the computed MDRI score, the harmonized harm score, per-dimension weighted normalized scores, the assigned risk category, the current alert state, and a recommended follow-up action or intervention payload. The structured response permits the calling external system to programmatically branch its own workflow on the returned risk category, alert state, and follow-up action, rather than treating system 100 as a write-only sink. In some embodiments, the API response also includes the configuration version identifier and timestamp used to produce the score, so that the receiving system can persist provenance.

In some embodiments, the processing system applies encryption-in-transit and encryption-at-rest protocols as security and compliance controls. This may help conform to different applicable healthcare and data protection regulatory requirements.

In some embodiments, the processing system further employs elastic compute resource scaling to dynamically adjust processing and storage resources based on incident volume and active user demand across the different healthcare systems. This allows the processing system to handle scaling performance when scaling the risk dimensions.

In some embodiments, the processing system is configured to integrate, via the application programming interfaces 112, with additional external healthcare equipment and operational systems including, without limitation: nurse call systems, medication dispensing cabinets, patient monitoring equipment, and staff and patient scheduling systems. In such embodiments, signals or events from these external systems are ingested as additional input data streams that may populate one or more dimensions of the risk model (for example, contributing to the operational impact dimension or detection/preventability dimension), and the structured response object returned by the API can be consumed by the same external systems to inform downstream workflows.

In some embodiments, the processing system is configured to be accessible through a microservice application with user interface (UI) elements embedded in the user device 130. In some embodiments, users can access the healthcare risk management server 100 through a browser application on the user device 130 to connect to network 120 using secure web connections with HTTPS (port 443) and TLS 1.2 encryption. Users can connect to the processing system by logging in to the microservice application. Users can access event files and MDRI user interface components from within the files. In some embodiments, data provided by users through the user interface components in the user device 130 is stored in a database within a dedicated database table.

In some embodiments, the risk data is stored according to a SaaS based deployment model using tenants or containers for each client. When the event reporting software is updated, the MDRI components would be updated accordingly.

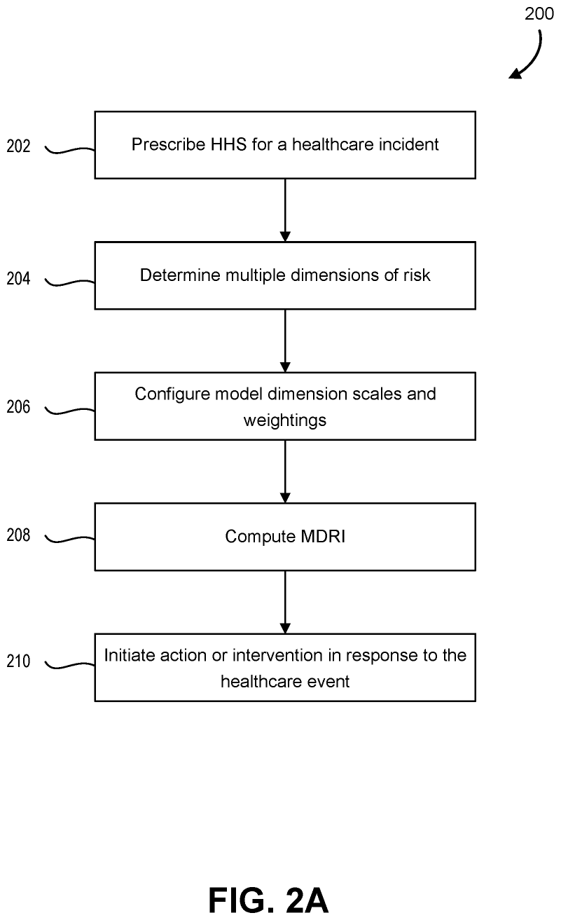

FIG. 2A is a process flow of a method 200 for healthcare risk management, according to some embodiments.

System 100 has one or more applications that utilize risk models 106 and store configurations for model dimensions and weights in memory 104.

At 202, system 100 receives input data about a healthcare event from I/O interface 136 as received by a user device 130 or from a plurality of input data streams from data sources 114. System 100 then establishes an HHS (or harm severity) for the healthcare event based on the received data.

In some embodiments, standardizing the input data involves receiving raw data from the plurality of input data streams in heterogeneous formats (e.g., HL7 FHIR resources from electronic medical record systems, CSV exports from laboratory information systems, JSON payloads from third-party reporting platforms, and XML messages from patient monitoring equipment) and applying a data-ingestion pipeline that performs format parsing, schema validation, unit conversion, temporal alignment, de-duplication, and mapping of source-specific field names to a canonical internal data schema. The resulting standardized input data can be uniform, machine-readable records in which each data element is expressed in a common representation (e.g., a normalised numeric value, a canonical code from a controlled vocabulary, or a timestamped textual narrative) that can be consumed by downstream computational stages of the processing system regardless of the originating source format. The standardization can ensure that the harmonized harm scores and dimensional risk scores computed from the input data are based on consistent, comparable data representations, improving the accuracy and reproducibility of the multidimensional risk index computation.

At 204, system 100 populates one or more dimensions of a risk model with standardized input data. In some embodiments, at least one of the plurality of input data streams can be from an event reporting system as a data source 114. Example dimensions include recurrence likelihood, operational impact, financial impact, staffing and workforce impact, clinical process impact, public perception or media impact, and regulatory compliance to drive decision-making and scoring of risks factors. System 100 provides flexibility and modularity such that users or clients can add dimensions that align with their specific care environment and strategic goals.

In some embodiments, the configurable risk model stored in memory 104 defines a set of active dimensions, normalization scale values for each active dimension, and importance weights for each active dimension. Each active dimension can represent a distinct risk category (for example, patient harm severity, likelihood of recurrence, operational impact, detection/preventability, or regulatory/organizational impact). The normalization scale values for each active dimension can include a minimum scale value and a maximum scale value that define the permitted scoring range for that dimension (for example, 0 to 5, or 0 to 100). The importance weights for each active dimension indicate the relative contribution of that dimension to the overall multidimensional risk index computation, with the sum of all importance weights across all active dimensions equalling one hundred percent. In some embodiments, the set of active dimensions is a proper subset of all available dimensions, such that one or more dimensions may be designated as inactive (disabled) in an organization's configuration and excluded from the computation. The configurable risk model may be versioned and stored with a configuration version identifier, and may be modified only by authorised users through role-based access controls. The processing system computes the multidimensional risk index value by, for each active dimension: dividing the entered dimension score by the maximum scale value to produce a normalised score; multiplying the normalised score by the importance weight for that dimension to produce a weighted normalised score; and aggregating the weighted normalised scores across all active dimensions to produce a total weighted normalised score constituting the multidimensional risk index value.

At 206, system 100 receives configurations for model dimensions and weights, and updating the configurations stored in the memory 104. System 100 can also receive updated risk models 106, or new risk model(s) 106.

In some embodiments, the harmonized harm scores are determined at least in part from the standardized input data. For example, the processing system may determine the harmonized harm score for a healthcare event by extracting, from the standardized input data, one or more of: a severity indicator reported by a frontline staff member, clinical outcome data (such as diagnosis codes, procedure codes, or length-of-stay metrics), and contextual metadata (such as the care setting, time of event, and patient acuity level). The extracted data elements can be evaluated against the HHS framework (levels 1 through 5) to produce or validate the harmonized harm score for the event. In some embodiments where a reporter selects an initial HHS value, the processing system uses the standardized input data to cross-reference the reported severity against objective clinical indicators, flagging discrepancies for review by an investigating official. The resulting harmonized harm score, whether determined directly from the standardized input data or validated against it, can serve as the clinical anchor for the multidimensional risk index computation.

System 100 receives information about a healthcare event in relation to a patient and organization. The configurations for model dimensions and weights may be specific to an organization. Each model may correspond to one organization. The model holds the HHS, the dimensions, the dimension scales, and the dimension weightings. The configurations for model dimensions and weights may be associated with metadata including an identifier for the organization, to distinguish or indicate what configurations and risk model to use for the organization that the incident relates to. The configurations for model dimensions and weights can indicate the dimensions used of the risk model, normalization min scale values and max scale values for each dimension, and weights for each dimension. In some embodiments, there can be multiple models for each organization that can be applied to, for example, different locations, care settings, events, and the like.

At 208, system 100 computes a multidimensional risk index for the incident based on the configurations for model dimensions and weights for the organization. For example, system 100 can compute the multidimensional risk index for the incident by normalizing each dimensions scale, applying an importance weighting to each dimension, and then aggregating each weighted dimension score to obtain a total weighted score. The configurations can indicate the weights and the normalization parameters.

Embodiments described herein integrate harm severity with additional dimensions such as the recurrence likelihood, operational impact, and regulatory compliance to drive decision-making and scoring of risks factors. System 100 can compute a score for each dimension and can then compute a normalized score for each dimension based on a min. scale value and a max. scale value. System 100 can then generate a normalized weighted score for each dimension based on the weight parameters for each dimension. System 100 can compute the MDRI by aggregating the normalized weighted scores for the dimensions.

At 210, system 100 initiates an action or intervention based on the dimension scores or multidimensional risk index. Interventions can be conducted at two levels of severity. The first level is if the risk of just one dimension (e.g., reputation) is high but all other dimension risks are low, then the MDRI score would likely not be high or concerning and a dimension-specific intervention could be utilized. The second level is if the risk indicates that several dimensions are involved with a high MDRI score. In this case, the same dimensional interventions would likely be utilized but the coordination of a high MDRI score would require more than one dimension subject matter expert to manage the issue or event.

System 100 is configured to generate visual elements at least in part corresponding to a portion of the multidimensional risk index, and the event.

System 100 is configured to generate improved interfaces and visualizations for selective display on display devices. Visualizations (such as trend lines, heat maps, and KPI cards, for example) are integral, interactive outputs of the system 100. The improved interfaces provide near real-time, interactive visual analytics to improve visual display systems and user experience.

FIG. 2B is a process flow for a method 250 of healthcare risk management with context-based risk surveillance according to some embodiments.

At 222, system 100 prescribes an HHS for a healthcare incident as described above with respect to FIG. 2A, and in method 250 receives the underlying input data through a bidirectional application programming interface configured to return structured response objects (computed MDRI score, risk category, alert state, recommended follow-up action, and configuration version identifier) to the calling system. Where the source organisation uses a non-HHS severity scale, system 100 maps the received severity onto the HHS using a per-tenant equivalency table. In some embodiments, system 100 invokes an artificial-intelligence-assisted scoring subsystem to propose an initial HHS or dimensional value for human confirmation, and records the value entered by the reporter as the initial reporter HHS and the value validated or adjusted by an investigating official as the system HHS that thereafter anchors all downstream computation and surveillance for the event.

At 224, system 100 determines multiple dimensions of risk and populates them with standardized input data as described above with respect to FIG. 2A, restricted in method 250 to the dimensions designated active under the tenant's current configuration version, and applies defined rules for missing or Not-Applicable dimensional values, including renormalisation across the remaining active dimensions, substitution of defined defaults, or computation of a partial MDRI that is flagged as such.

At 226, system 100 configures model dimension scales and weightings as described above with respect to FIG. 2A, and additionally receives configurations for color-coding ranges, smoothing parameters, control-band coefficients, and intervention rules, and updates the configurations stored in memory 104 under per-tenant version control, with each configuration record assigned a configuration version identifier together with audit metadata and with weighting changes restricted by role-based access control and optionally previewed against historical event data prior to activation. Before any MDRI computation under a configuration version, system 100 validates that each dimensional score falls within the permitted range for that dimension, that each dimension being scored is designated active under the configuration version, and that the active dimension weights sum to one hundred percent. On any validation failure, system 100 rejects the input through the bidirectional application programming interface.

At 228, system 100 computes a multidimensional risk index for the incident based on the configurations for model dimensions and weights for the organisation as described above with respect to FIG. 2A, and persists, with the resulting MDRI record, the configuration version identifier under which the score was computed, so that any historical signal may be re-rendered against the configuration that was active when the underlying event was scored.

At 230, system 100 fits a smoothing model to the tenant's historical, normalised MDRI series to produce an expected risk trajectory and dynamic control bands about the trajectory. In some embodiments, the smoothing model is a polynomial regression of the form y=β0+βx+β2x²+ . . . +βnxⁿ+ε, where y is the expected risk value, x is the time or reporting period, β0 is the intercept, β1 through βn are coefficients fitted from the tenant's historical data, n is the polynomial degree, and ε is the residual variation. Alternative configurable smoothing methods including rolling averages, moving averages, exponentially weighted moving averages, and statistical-process-control methods such as Shewhart and CUSUM may be selected on a per-tenant basis.

At 232, system 100 compares the current MDRI value computed at 208 against the expected risk trajectory and the dynamic control bands produced at 230, and stratifies the current activity as nominal, watch, sentinel, or drift based on the band crossings.

At 234, system 100 initiates an action or intervention in response to the stratification at 232, by auto-prefilling an Investigator's Form with the contributing dimensional values, the configuration version identifier, the trajectory parameters, and the band crossings, by routing the Form to the responsible role under role-based access control. Structured work-items are dispatched through an Intervention Engine in accordance with the tenant's intervention rules and the Grandparent>Parent>Child (G>P>C) task hierarchy.

System 100 further writes investigator inputs and intervention outcomes, including any update to the system HHS or dimensional values, any annotation of the dashboard, any tagging of the signal as actionable or not actionable, and any documented outcome of the intervention, back to the per-tenant store. Re-computation of the MDRI may be triggered upon any change to the manager-confirmed HHS, a dimensional value, added context, or the active configuration version, thereby feeding longitudinal intervention-effectiveness analytics and completing the closed-loop feedback path of method 250.

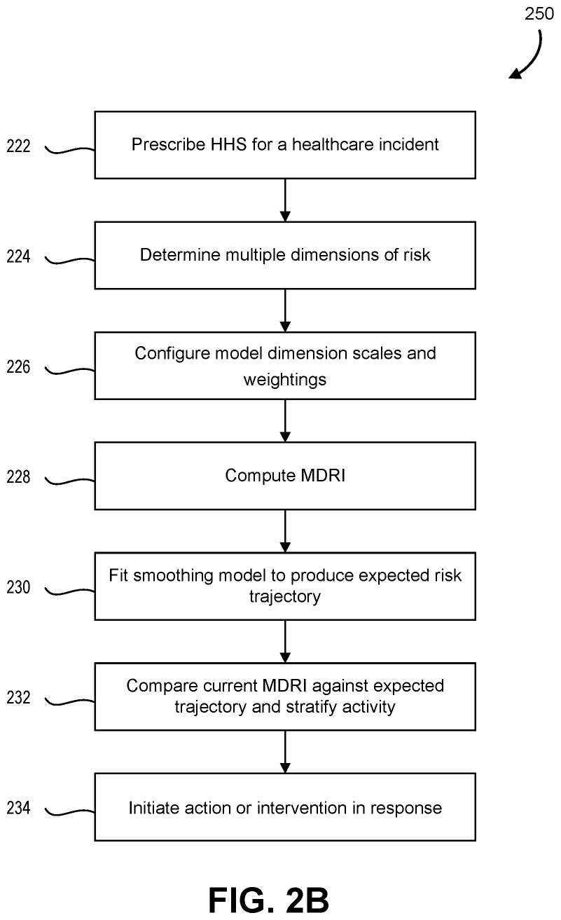

FIG. 3 is a schematic of data flow for healthcare risk management using Harmonized Harm Score (HHS), Multi-Dimensional Risk Index (MDRI) and Context-Based Risk Surveillance (CBRS) systems, according to some embodiments.

Embodiments described herein operate through a multi-phase process to proactively manage the scoring of events in near real time within healthcare organizations and entities. In the first phase, an incident occurs, and the incident is reported and filed. Then, the incident is categorized using a Harmonized Harm Score (HHS) that measures the direct patient impact (e.g., no harm to death), ensuring a standardized severity assessment. In some embodiments, a user (e.g., risk manager) reviews the initial incident and can adjust and validate the HHS for accuracy and consistency.

In the second phase, multiple dimensions of risk are captured, computed and analyzed, providing a more nuanced understanding of organizational impact. These dimensions and factors are then combined into the MDRI score, which helps risk managers prioritize interventions based on severity and potential recurrence. The combination of the HHS dimension score (which denotes patient harm) with the concept of additional harm dimensions scores to generate the MDRI and also the individual dimensions scores drives both interventions and systemic changes.

Embodiments described herein operate through a structured three-step process to obtain the MDRI score for an incident. First, the incident is categorized using a Harmonized Harm Score (HHS) that measures the direct patient impact (e.g., no harm to death), ensuring a standardized severity assessment. The assessment can be provided as an interface e.g. at user device 130 to capture standardized input data, for example. Next, multiple dimensions of risk are captured, computed and analyzed, providing a more nuanced understanding of organizational impact. In some embodiments, the multiple dimensions of risk include recurrence risk, operational impact, detectability/preventability, and regulatory compliance. If a particular dimension of risk has a high score, dimension-specific interventions or actions can be initiated to address the risk. For example, a high reputational risk dimension score could result in specific dimension interventions or related actions even if the MDRI score is low enough to not initiate any further review. Finally, these factors are combined into the MDRI score, which helps risk managers prioritize interventions based on severity and potential recurrence. The combination of the HHS dimension score (which denotes patient harm) with the concept of additional harm dimension scores to generate the MDRI and also the individual dimensions scores drives both interventions and systemic changes.

In some embodiments, there is a third phase of leveraging the outputs of the MDRI to undergo CBRS, which offers dynamic analysis with forecasting capabilities and risk insights specific to a healthcare entity and its unique patient population. CBRS also integrates organizational priorities and resource allocation into the monitoring and response mechanisms, allowing for configuration to each organization's contexts and priorities.

In some embodiments, there is a fourth phase involving interactive investigation within the CBRS. For example, users can fill out an Investigator's Form after a statistical or context-sensitive deviation is detected to transition data signals into actionable risk intelligence through a human-in-the-loop layer. The Investigator's Form would function as a contextual investigation and interpretation tool. Examples of detected statistical or context-sensitive deviations include a spike in wrong-patient medication errors during shift change, a rise in patient falls on night shifts, an outlier in procedure related complications after a process change, and the like. The visualization output is adjusted based on the contextual investigation with on-going and on-demand executive reporting.

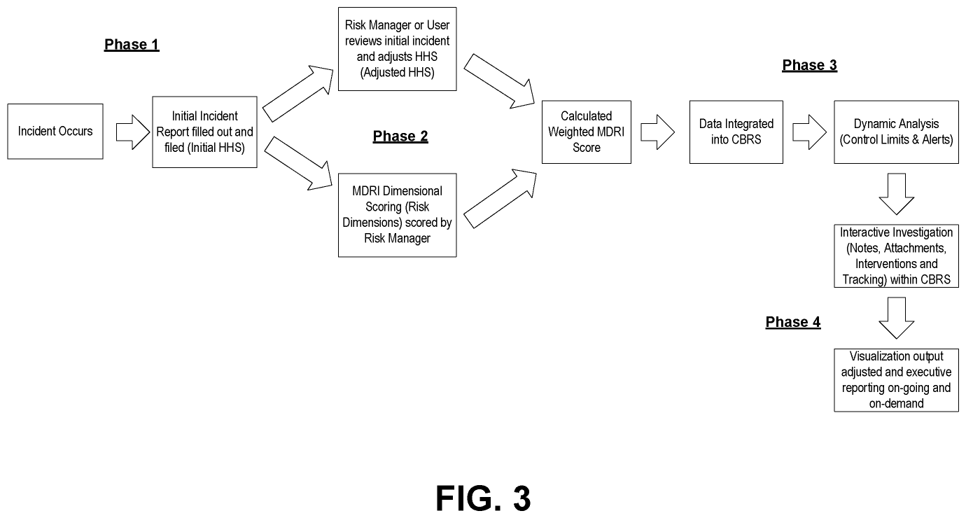

FIG. 4 is a schematic of data flow 400 for healthcare risk management according to some embodiments that involve MDRI. The data flow 400 may be for a cloud based healthcare operations system, for example. The components shown in the data flow 400 can form part of a larger operational system of operational applications for healthcare risk management. For example, an operational application system can include MDRI, HHS, and CBRS data capture and processing. The example data flow 400 diagram includes different example operational applications for risk and safety, compliance management, contract management, provider management, policy management, credentialing management, data management, workforce management, and patient satisfaction. The different operational applications can provide data for MDRI.

The example data flow 400 can enable cloud-based, standardized data capture and processing across all operational domains, allowing analysis to be conducted departmentally and at the enterprise level. Each risk dimension can have a configurable scale (e.g., 0-5, 0-100), and the original scores are divided by the maximum of their corresponding scales to produce a normalized value between 0 and 1. The normalized values allow all dimensions to be compared and weighted consistently. The example data flow 400 can support trend analysis dashboards with visual features that highlight recurring issues to help visualize risk in real-time and guide proactive interventions. Example visual features provided on trend analysis dashboards include MDRI score trend lines with control bands, stacked bar charts for dimensions, heatmaps for incident concentration, interactive alerts (e.g., red flags, intervention trackers), and a CBRS Dashboard and Context Aware Display. In some embodiments, the data flow 400 can be used to automatically trigger alerts for high-likelihood incidents to notify leadership or process improvement teams.

### Phase 1: Harmonized Harm Score (HHS)

Embodiments described herein operate through a structured multi-step process to obtain the MDRI score for an incident. For example, embodiments described herein implement one or more operations to categorize the incident using the Harmonized Harm Score (HHS), ensuring a standardized severity assessment.

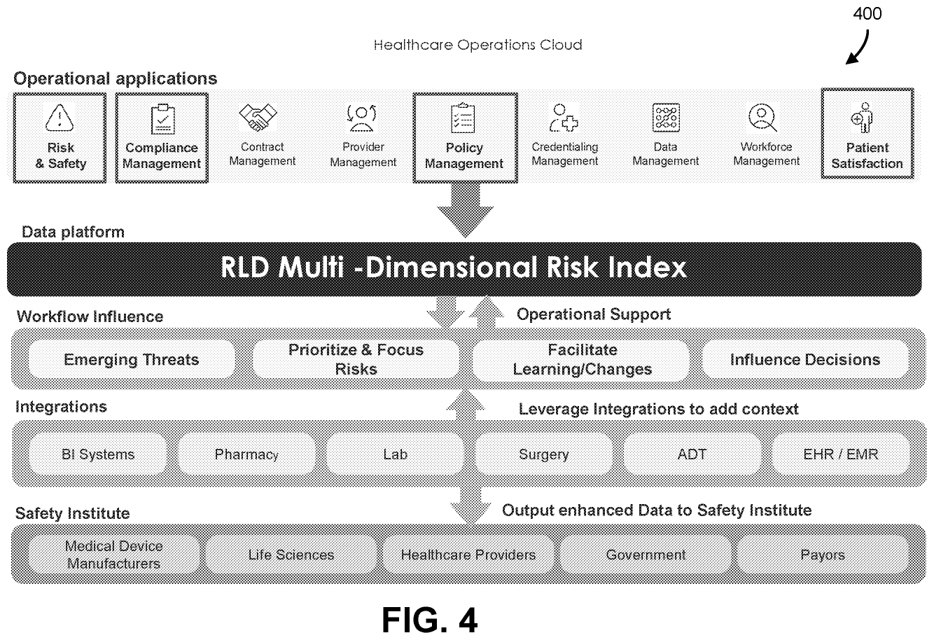

FIG. 5 is an example table of harm score levels for categorizing incidents based on harm caused or the potential for harm, according to some embodiments.

Scoring systems are met with a number of challenges, including complexity and confusion, inconsistent data capture, form abandonment, non-initiation, prolonged completion times and organizational inefficiency. Some scoring frameworks are cumbersome, numerous, and lead to frequent misinterpretation and incorrect assessments of patient harm levels. Variability in user inputs causes discrepancies in the classification of events, undermining the reliability of the data collected, which in turn can result in the initiation of incorrect workflows which can result in delays in event reviews. Furthermore, the perceived difficulty and complexity of completing harm assessments or reporting events can lead users to only partially complete or not complete forms at all. Users spend excessive time navigating scoring processes, which detracts from time spent on patient care and analysis, and the inability to easily align harm scoring systems across departments or institutions hampers benchmarking and systemic safety improvements.

The Harmonized Harm Score (HHS) provides an improved standardized scoring framework designed to integrate seamlessly with existing safety reporting systems. The HHS provides a reliable and consistent way to capture the patient harm dimension of events as it is commonly the heaviest weighted or most important dimension for decision-makers. The HHS provides a clear, intuitive methodology for assessing harm consistently across platforms and organizations.

TABLE 1
below shows a comparison between the HHS scoring system levels and score levels of
other risk severity scoring systems, determined using harm severity equivalency.

| | | AHRQ | | | | | CPSI | | |
|---|---|---|---|---|---|---|---|---|---|
| Harmonized | NCC | Common | WHO | VHA Harm | | NHS | Incident | ACSQHC | FDA |
| Harm Score | MERP | Formats | ICPS | Scale | IHI GTT | NRLS | Analysis | SAC | MedWatch |
| Level 1: No | A-D | No harm | No harm | No Harm (Close | E (temporary no | No harm | No harm | SAC 4 | Minimal/no |
| Harm | | | | Call) | intervention) | | | | intervention |
| Level 2: | E | Mild | Minor | Minor harm | F (temporary | Low | Minimal or | SAC 3 | Minor |
| Minor Harm | | temporary | temporary | | minor | harm | mild harm | | intervention |
| | | harm | harm | | intervention) | | | | required |
| Level 3: | E-F | Moderate | Moderate | Minor/moderate | F (temporary | Moderate | Moderate | SAC 2 | Medical |
| Moderate | | temporary | temporary | harm | hospitalization) | harm | harm | | intervention or |
| Harm | | harm | harm | | | | | | hospitalization |
| Level 4: | G-H | Severe | Severe | Permanent | G/H (permanent | Severe | Severe | SAC 1 | Extensive |
| Severe Harm | | permanent | permanent | harm/extensive | harm or life- | harm | permanent | | medical |
| | | harm | harm | intervention | sustaining) | | harm | | intervention, |
| | | | | | | | | | permanent |
| | | | | | | | | | impairment |
| Level 5: | I | Death | Death | Death | I (Death) | Death | Death | SAC 1 | Death |
| Death | | | | | | | | | |

Using the HHS provides standard categories and definitions to minimize user confusion. The single, harmonized structure can align with other reporting standards, including National Patient Safety Goals (NPSG), Serious Reportable Events (SREs), TJC Sentinel Events, NCC MERP, AHRQ, and the like, to facilitate seamless data integration and comparison. Embodiments described herein are developed based on user feedback to improve ease of use, reducing the cognitive load on reporter users and simplifying the scoring process to reduce confusion, misclassification and time-to-completion for users. Embodiments described herein establish a unified language for harm classification, enabling accurate and comparable data across departments and organizations. Embodiments described herein remove barriers related to complexity and facilitate internal and external benchmarking through standardized metrics, improving the ability to identify trends and drive quality improvement by identifying high-risk areas efficiently and allocating resources where they are most needed. Furthermore, embodiments described herein directly contribute to actionable insights that reduce harm and improve patient safety by improving the accuracy and usability of harm scoring, allowing users to proactively address incidents with high recurrence or severe consequences.

In some embodiments, the harm-severity equivalency mapping reflected in Table 1 and used by the processing system to translate or map third-party harm classifications into the HHS framework is constructed from a plurality of recognized harm-severity reference frameworks, including without limitation: the National Coordinating Council for Medication Error Reporting and Prevention (NCC MERP) Index for Categorizing Errors; the Agency for Healthcare Research and Quality (AHRQ) Common Formats; the Canadian Patient Safety Institute (CPSI) Incident Analysis framework; the World Health Organization International Classification for Patient Safety (WHO ICPS); the Veterans Health Administration (VHA) Harm Scale; the Patient Safety Reporting System (PSRS); the Institute for Healthcare Improvement (IHI) Global Trigger Tool (GTT); the United Kingdom National Health Service National Reporting and Learning System (NHS NRLS); the Australian Commission on Safety and Quality in Health Care (ACSQHC) Severity Assessment Code (SAC); and the United States Food and Drug Administration MedWatch program. In some embodiments, the processing system stores, per organization, a mapping table that translates an incoming third-party severity code into the HHS 1-5 scale, so that organizations electing to retain their existing harm classification can still feed data into MDRI computation and CBRS surveillance through a normalized HHS value.

Harm scores allow for risk prioritization for immediate action, assessment standardization, estimation of incident impact, reporting and learning from adverse events, and proactive risk assessment.

Harmonizing the harm score across reporting systems ensures consistency in how adverse events are identified, classified, and addressed, ensuring a unified language is used for patient safety. This alignment reduces variability in data interpretation, improves the accuracy of risk analysis, and enables benchmarking across organizations. In particular, the HHS provides a score accurately capturing the patient harm dimension of risk that can be used to compute an accurate MDRI score. Furthermore, this can simplify workflows for frontline staff, enhances transparency for stakeholders, and strengthens regulatory compliance. By focusing on a standardized approach, healthcare organizations can more effectively allocate resources to high-priority safety initiatives, ultimately reducing harm and improving patient outcomes.

The HHS determination initiates when an incident is reported and filed. In some embodiments, the event is reported through a Risk Event Form by a user. The registrant of the event has HHS or an equivalent client classification system on the Incident Form. The HHS can be adjusted or validated by an investigating official for accuracy and consistency. The adjusted HHS then serves as a foundational data point for blending in MDRI calculations for validation of the harm severity to the patient and normalization of data for MRDI blending. In some embodiments, MDRI calculations can be used for patient safety organization (PSO) consumption, where PSOs are specialized entities designed to improve patient safety and healthcare quality through the collection, analysis and sharing of patient safety event data. In some embodiments, users can choose to remain with using their choice of harm severity scoring to be mapped to the MDRI framework instead of adopting the HHS score.

In some embodiments, data visualizations can be outputted to users or clients for further analysis.

In some embodiments, the HHS is captured in two stages. In a first stage, an initial reporter (for example, a frontline staff member completing the Risk Event Form at or near the point of incident occurrence) selects an initial HHS value. The initial reporter HHS value functions as a starting point only and is provisionally stored as the “reporter HHS.” In a second stage, an investigating official, risk manager, or other authorized reviewer reviews the incident narrative and supporting context and either confirms the reporter HHS or overrides it with an adjusted value. The confirmed or adjusted value is stored as the “system HHS” (also referred to herein as the manager-confirmed HHS). The processing system uses the system HHS, rather than the initial reporter HHS, as the clinical anchor for downstream MDRI computation and Context-Based Risk Surveillance. The processing system retains both values in the database, with timestamps and user credentials for each, so that variance between reporter HHS and system HHS can itself be analyzed (for example, to identify under-scoring or over-scoring patterns by reporter cohort, location, or shift).

In some embodiments, the position of the HHS field within the Risk Event Form is configurable per organization. The HHS field may be presented at the front of the form (for immediate triage and to drive downstream conditional fields), at the back of the form (so that the HHS field functions as a flow filter, gating submission only after upstream contextual fields have been completed), or at an intermediate position. In some embodiments, the processing system presents the HHS selection as a low-cognitive-load interaction (for example, as a pop-up, a stepped slider, or a visual scale with plain-language anchors) so as to reduce reporter abandonment and to standardize the captured value at the moment of report.

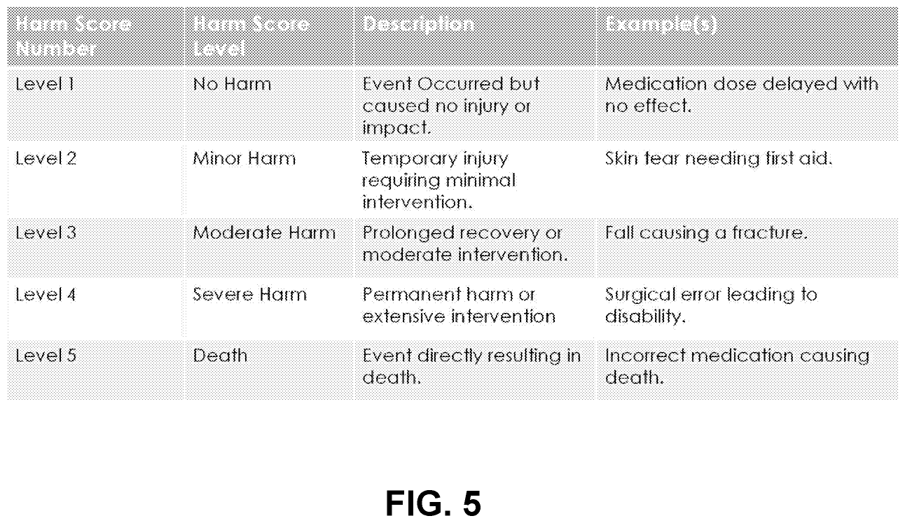

FIG. 6 shows a stacked bar chart visualization of example incidents categorized based on risk severity on the HHS scale, according to some embodiments. Each segment of the horizontal bar graphs corresponds to data from one quarter of a year. In FIG. 6, the majority of the number of quarterly incident occurrences is in the HHS 1 level categorized as no harm with approximately 420 occurrences in total. In comparison, the number of quarterly incident occurrences that are in the HHS 5 level categorized as death is very low with approximately 5 occurrences in total.

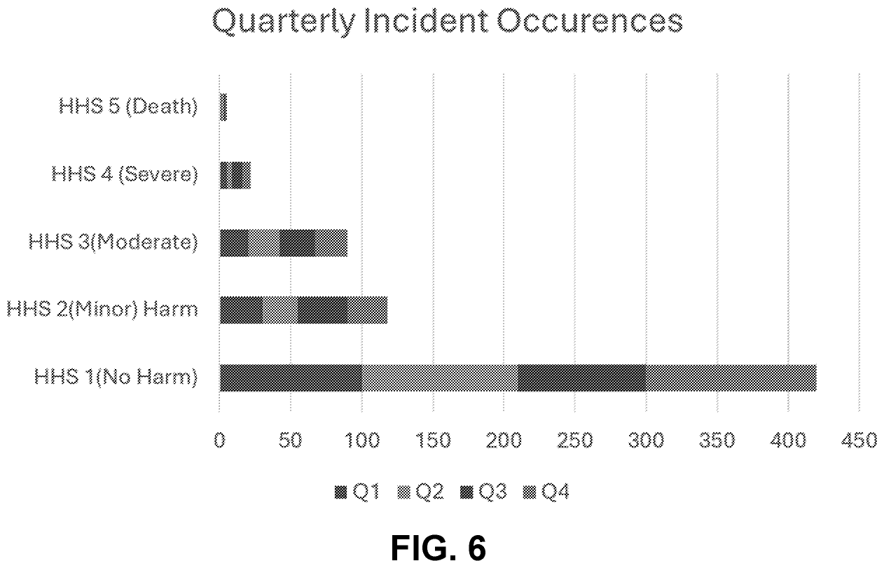

FIG. 7 shows a pie chart visualization of example events or incidents categorized based on risk severity on the HHS scale, according to some embodiments. Similar to FIG. 6, the number of events categorized as HHS 1 level or no harm is a significant majority compared to the number of events categorized as the other HHS levels. The number of events in each category decreases with the harm severity of each HHS category.

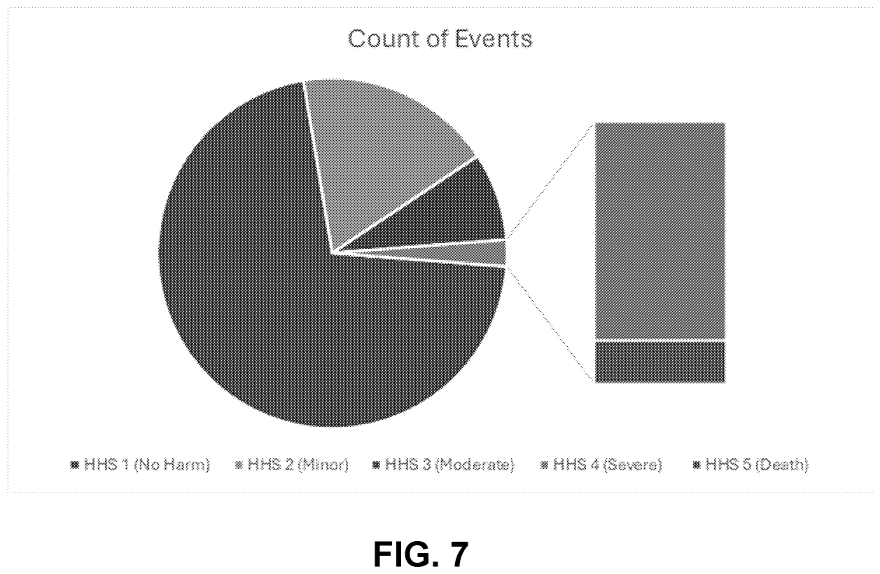

FIG. 8 shows a line graph visualization of example incidents over time categorized based on risk severity on the HHS scale, according to some embodiments. This visualization allows users to observe and analyze trends and differences in the amount of incidents of each HHS category level for each month over time. For example, according to the graph in FIG. 8, the amount of HHS 1 level incidents in April that resulted in no harm decreased by approximately 20 in comparison to the previous month of March.

### Phase 2: Dimensions of Risk and the MDRI Score

Next, multiple dimensions of risk are captured and analyzed, providing a more nuanced understanding of organizational impact. Examples of the multiple dimensions of risk include the HHS for standardized scoring of patient harm severity (HHS), likelihood of recurrence for risk of an event happening again (R), operational impact for assessing effect on hospital workflow and resource allocation (O), detectability and preventability for how easily the risk can be identified and mitigated (D), and regulatory compliance and organizational impact for compliance, legal, and reputational concerns (I). The scoring scale and importance weighing for each of the risk dimensions can be determined and configured to reflect the organization's or entity's key priorities and concerns. In some embodiments, scores for each dimension of risk are determined manually by a Risk Manager or designated reviewer based on the narrative and context of the event. In some embodiments, scores for each dimension of risk are assessed using automated scoring with electronic health record data or artificial intelligence tools. In some embodiments, scores for each dimension are generated based on prior events that were similar. In some embodiments, scores for each dimension are generated by consensus where multiple Risk Managers score dimensions and averages are taken.

In some embodiments, dimensional scores and the HHS are populated using one or more artificial intelligence (AI) or machine-learning components of the processing system. In particular, the processing system may include a prompt-engineering subsystem that constructs structured natural-language prompts from the incident narrative, the captured HHS, and contextual metadata (e.g., unit, time of day, staffing ratio at the time of the event), and submits the prompts to a large-language-model (LLM) inference service to produce a proposed HHS value, proposed dimensional scores, and a written rationale for each proposed value. The proposed values are presented to the risk manager or investigator as suggestions, not as final values, while the manager-confirmed HHS and dimensional scores remain the authoritative inputs to MDRI computation. The processing system logs the prompt template version, the model identifier, the model temperature or other inference parameters, and the manager's accept/override decision for each AI-proposed value, so that AI-assisted scoring is auditable and the divergence between AI-proposed and human-confirmed values can be analyzed over time. In some embodiments, AI-assisted scoring is gated behind role-based access controls and is disabled by default for organizations that have not opted in.

Further, these factors are combined into the MDRI score, which helps risk managers prioritize interventions based on severity and potential recurrence. The MDRI evaluates multiple dimensions of harm to assess the impact that any given event may have on a healthcare organization. The combination of the HHS and severity index (which denotes patient harm) with the concept of additional harm dimensions to generate the MDRI and also the individual dimensions scores drives both interventions and systemic changes.

A harm scale that evaluates event severity solely based on patient harm can have significant limitations, particularly in identifying risks, preventing future incidents, ensuring comprehensive safety, and identifying broader underlying risks. When you look at severity only through the lens of patient harm, it does not accurately reflect the impact that event could have on the broader organization as it pertains to other dimensions of risk, including legal risk, reputational risk, operational risk, likelihood of recurrence, detection/preventability and others. Most created harm scales only consider two or three variables in their scoring. This leads to a lack of understanding of the true impact that an event might have on an organization or entity. Below are some harm scores that might seem familiar.

For example, the ACSQHC SAC score relies only on patient harm severity and likelihood. As another example, the CPSI Incident Analysis score only relies on severity, likelihood and detectability and the HPI score only relies on actual harm and potential harm factors.

The MDRI is calculated as the sum of the weighted normalized scores (WNS) of all dimensions (including the HHS). The MDRI combines the HHS derived from patient harm severity with a total weighted score (TWS) computed through the captured dimensions. In some embodiments, the MDRI can be calculated on a scale of 0-100, as per below:

$$MDRI = 100 \times [ WNS ( HHS ) + WNS ( TWS ) ] = 100 \times [ WNS ( HHS ) + WNS ( L ) + WNS ( 0 ) + WNS ( D ) + WNS ( R ) ] \qquad ( 1 )$$

where WNS represents the weighted normalized score for a dimension, HHS is the harmonized harm score, and L, O, D, and R are additional dimensions of risk.

In some embodiments, a MDRI score is given a numeric score and color to represent the level of harm that an event has on an organization across the various dimensions of risk. For example, for some organizations, a MDRI score within the range of 0-33 represents an incident of low organizational harm which can be managed systemically or by quarterly review. In some embodiments, a MDRI score within the range of 34-66 represents an incident of medium organizational harm that requires targeted interventions (perhaps at the individual dimension level). In some embodiments, a MDRI score within the range of 67-100 represents an incident of high organizational harm that requires immediate action.

Examples of immediate actions include alerts, notifications, critical communication audit, escalation protocol, and discharge summary revision, and the like either at the individual dimension or MDRI level. For example, critical communication audit can involve performing a root cause analysis to analyze the communication breakdown and identify gaps in the lab-to-clinician notification process. For example, an escalation protocol can involve implementing a backup system where results not acted upon by the primary contact are escalated to another clinician or team leader. For example, a discharge summary revision can involve adding a dedicated Test Pending at Discharge (TPAD) section in discharge summaries to list pending results and specify follow-up responsibilities.

Examples of other actions, alerts, and notifications that may be triggered by an MDRI score include red flag alerts when the MDRI score crosses +2 Sigma threshold, automated emails to unit managers, launch of an Investigation Form or root cause analysis task, color-coded dashboard updates, auto-assignment of intervention checklists, and the like, which all promote accountability and timely follow-up.

In some embodiments, the proposed system can integrate with healthcare event reporting software.

In some embodiments, further additional dimensions can be added for forecasting analytics.

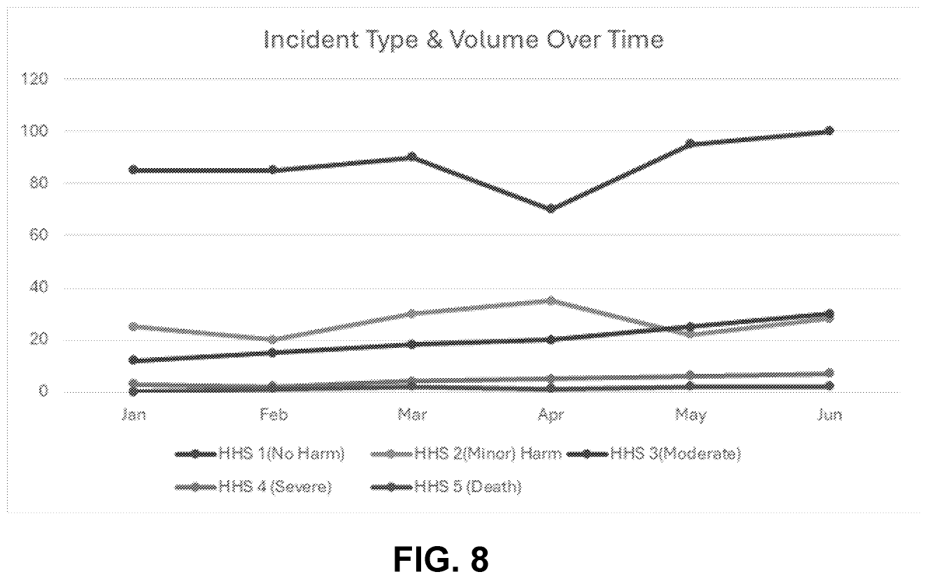

FIG. 9 shows an example table of weighted risk scores, according to some embodiments.

System 100 manages configurations for model dimensions and weights for different organizations. The configurations for model dimensions and weights may be associated with metadata including an identifier for the organization, to distinguish or indicate what configurations and risk model to use for the organization that the incident relates to.

The configurations for model dimensions and weights can indicate the dimensions used for the risk model, minimum scale values and maximum scale values for each dimensions, and importance weights for each dimension. The minimum and maximum scale values define the scoring range (e.g., 0-5, 0-100). When evaluating the dimensions, the user assigns an original dimension score that sits within the range of the corresponding scale to each dimension. The original scores are normalized against the maximum scale values of their scoring range, as shown below:

$$Dimension Normalized Score = \frac{Original Dimension Score}{Max Scale Value for that Dimension} \qquad ( 2 )$$

Given the variability in the scales, each dimension's score is normalized to a percentage or a normalized score number that is within the range of 0-1. For the example risk scores in FIG. 9, each component, including HHS, is normalized according to Equation 2 and weighted independently before being aggregated into the final MDRI score of 68. The example risk scores of HHS, likelihood of recurrence, operational impact, detection/preventability, and reputational/legal/compliance are normalized to 0.75, 0.20, 1.00, 0.60 and 1.00, respectively.

In some embodiments, the scale for the risk dimensions can be determined at implementation and re-scaled accordingly depending on organizational priorities.

Embodiments described herein provide an interface for configuring or customizing the MDRI to allow for organization-specific alignment and priority-driven decision-making. For example, the interface can enable configuration of the dimensions used for the MDRI. As another example, the interface can enable configuration of the weightings and scoring scales for each dimension used for the MDRI.

Normalized scores are customized and weighted based on organizational priority to emphasize their relative importance in the MDRI calculation, with the sum of the weights equalling 1 or 100%. Being able to adjust the weightings for each dimension allows for independent and systemic action on HHS, individual dimensional scores and the MDRI score, providing flexibility for users to control the importance weightings on all dimensions (including HHS). The proposed system scales easily to support additional dimensions that may become important to different users. Furthermore, organizational harm or impact can still be identified even when there is low or not patient harm. The dimension weighting is applied to the dimensions' normalized scores to calculate a weighted normalized score for the dimension, as shown below:

Weighted Normalized Score=Dimension Weighting×Dimension Normalized Score (3)

In some embodiments, the processing system performs a plurality of validation operations on the configurations and the entered scores prior to, and as a precondition for, computing the MDRI. The validation operations include: (i) verifying that each entered original dimension score is within the permitted minimum-to-maximum range defined for that dimension by the active configuration (for example, rejecting an entered score of 7 where the configured scale is 1-5); (ii) verifying that each dimension being scored is currently active in the organization's approved configuration, and rejecting or quarantining scores entered against a deactivated dimension; (iii) verifying that the importance weights of all currently active dimensions sum to 100% (or to 1.0, depending on representation) before any configuration is permitted to be activated for production use; and (iv) verifying that the configuration carries an organization identifier and a configuration version identifier. A configuration that fails any of these validation operations is not permitted to be used to compute a production MDRI score, and the processing system returns a structured validation error to the calling interface or external system identifying the failed check.

In some embodiments, the processing system applies defined rules for handling missing values or values flagged as “Not Applicable” (N/A) for one or more dimensions. The rules may include, in different configurations: (a) excluding the missing or N/A dimension from both the numerator and the denominator of the weighted aggregation, with the remaining active-dimension weights renormalized to sum to 100% prior to aggregation; (b) substituting a configured default value (for example, the dimension's neutral midpoint) for the missing dimension while preserving the original weight distribution; or (c) refusing to compute a final MDRI and instead returning a partial-MDRI flag together with the dimensions actually scored. The selected rule is recorded with the resulting MDRI calculation so that downstream analytics and CBRS surveillance can distinguish full-MDRI scores from rule-imputed or partial-MDRI scores and can avoid drawing inconsistent comparisons across events with differing dimensional coverage.

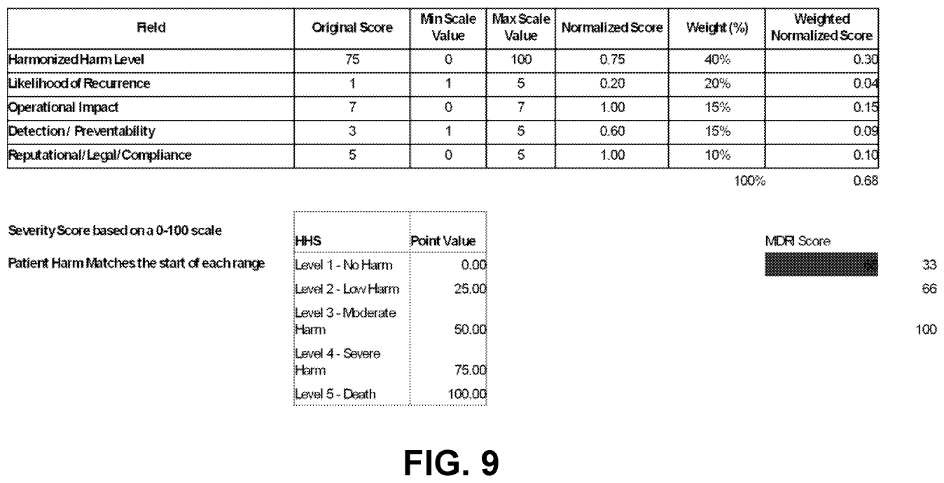

FIG. 10A-10B show tables of example weighted risk scores with different weightings for each dimension of risk, according to some embodiments.

Different weight configurations prioritize different dimensions of risk. Embodiments described herein provide an interface (e.g. at user device 130) that allows organizations to adjust weights based on their unique contexts and situations. MDRI scoring can be tested and refined during initial implementation to ensure data scoring consistency and proper threshold notifications. Changing weightings too frequently can compromise accuracy of reporting and data interpretation. Frequent changes to weights undermine trend analysis and make longitudinal data unreliable. In some embodiments, it is recommended that users review and lock weight settings for at least 6-12 months unless major operational or regulatory changes require adjustment.

In some embodiments, the processing system applies a governance layer to changes in dimensional weights and to other configuration changes. Weight changes are auditable (each change is timestamped and user-attributed), are role-restricted (only an administrator role can submit a weight change, and in some embodiments a second authorized user must approve the change before it becomes effective), and are versioned. In addition, prior to activation of a proposed weight change, the processing system is configured to execute an impact-preview simulation: the proposed weights are applied retrospectively to a configurable historical window of incidents, and the processing system surfaces, in the configuration interface, the resulting changes (for example, the count of incidents whose risk category would shift, the change in the daily average MDRI, and the change in the degree of exceedances over the window) before the administrator commits the change. The administrator may then either commit, modify, or discard the proposed change. This impact-preview mechanism allows weight tuning to be performed with an explicit understanding of its surveillance consequences before going live.

A higher weight for HHS ensures patient harm remains a priority. A higher weight for recurrence ensures frequent risks are surfaced. Adjusting weights allows configuration or customization based on organization priorities or preferences. System 100 stores the configurations or customization for different users.

In FIG. 10A, a balanced weighting approach is chosen where: HHS=20%, L=20%, O=20%, D=20%, and R=20%, resulting in a calculated MDRI score of 71 using Equation 1. In FIG. 10B, a high dimension weighting approach is used where: HHS=5%, L=40%, O=5%, D=40% and R=10%, resulting in weighted normalized scores: WNS(HHS)=0.04, WNS(L)=0.08, WNS(O)=0.05, WNS(D)=0.24, and WNS(R)=0.10, which are summed together and scaled to a score between 0-100 for a calculated MDRI score of 51.

As another example, there may be a weight configuration where: HHS=40%, L=20%, O=15%, D=15%, and R=10%. Each approach changes the MDRI score output. An MDRI score can be recomputed and updated in response to configuration changes.

In some embodiments, the processing system recomputes the MDRI for an event in response to one or more defined re-computation triggers, including: (i) a change in the manager-confirmed (system) HHS; (ii) a change in any dimensional value associated with the event (whether by the original investigator, by a collaborating subject-matter expert, or by an AI-assisted re-scoring); (iii) the addition of new contextual information to the event record (for example, an attached witness statement, a linked laboratory result, or a follow-up note); and (iv) the activation of a new organization-approved configuration version (for example, a change in dimension weights or in the active-dimension set). On each re-computation, the processing system writes a new MDRI record linked to the event, retaining prior MDRI records for audit and trend analysis, and may initiate, escalate, or de-escalate downstream actions and CBRS alerts based on the recomputed value.

In some embodiments, to determine if the MDRI score is a concerning number or not, the organization or entity will need to define what is considered to be low, moderate or high impact ranges, and color code them appropriately. In this way, color coding of the MDRI scores is varied to be specific and customized to each entity or organization's own priorities and needs. Example color coding scales include fixed ranges (e.g., there are three ranges, one each for red, yellow and green) and gradient ranges (e.g., green fades into yellow which fades into red with 0 on the left end being green and 100 on the right end being red).

In a practical exemplary scenario, there can be an incident of test pending at discharge (TPAD) resulting in septic shock. For example, a patient is hospitalized after a fall and shortness of breath. Diagnosed with pneumothorax, a chest tube was placed, and the patient improved clinically. Discharge of the patient occurred two days later, despite a pleural fluid culture result pending. On the day of discharge, the culture returned positive for gram-positive cocci, but the attending physician could not be reached, and no escalation occurred. Two days later, the patient was admitted to another hospital in septic shock, requiring chest tubes, IV antibiotics and a PICC line for long-term treatment. The patient eventually recovered but filed a claim against the hospital.

TABLE 2
below shows representative scoring for additional
risk dimensions for this example.

| | | Scoring | |
|---|---|---|---|
| Dimension | Score | Scale | Rationale |
| Harm Score (HS) | 75 | 0-100 | Life-threatening septic shock |
| | | | requiring invasive treatment. |
| Likelihood of | 5 | 0-5 | TPAD-related communication |
| Recurrence (R) | | | breakdowns are well- |
| | | | documented as frequent. |
| Operational | 5 | 0-7 | Significant resource utilization |
| Impact (O) | | | at the secondary hospital due |
| | | | to delayed diagnosis. |
| Detection/ | 3 | 0-5 | Preventable with better |
| Preventability (D) | | | closed-loop communication |
| | | | and follow-up protocols. |
| Organizational/ | 5 | 0-5 | High exposure to regulatory |
| Regulatory | | | and reputational risk due to |
| Impact (I) | | | systemic flaws. |

The first step is to establish the HHS level of the incident. In this example, the delayed follow-up on the positive culture resulted in septic shock, a life-threatening condition requiring significant intervention. Therefore, the patient harm level would be severe harm, which is equivalent to HHS Level 4 and score 75/100.

The second step is to assess multiple dimensions of risk. First, the likelihood of recurrence can be considered. In this example, TPADs are common, and studies show that typically many require action, with breakdowns in communication being a frequent contributor. Therefore, the assessment of likelihood of recurrence can would be frequent (e.g., score of 5 out of 5). Next, operational impact can be considered. In this example, the failure to act on the TPAD resulted in prolonged hospitalization, costly interventions, and resource strain. Therefore, the assessment of operational impact would be major impact (e.g., score of 5 out of 7). Next, detection/preventability can be considered. In this example, the missed communication could have been prevented with closed-loop communication systems, escalation protocols, and proper documentation. Therefore, the assessment of detection/preventability would be moderately detectable (e.g., score of 3 out of 5). Next, organizational/regulatory impact can be considered. In this example, the incident highlights systemic flaws in TPAD management and critical test result communication, exposing the organization to regulatory and reputational risks. Therefore, the assessment of organizational/regulatory impact would be high impact (e.g., score of 5 out of 5).

The third step is to normalize the harm scores and calculate the normalized score for each dimension by dividing each dimension score by its corresponding maximum scale value. Then, the importance weighting for each dimension is applied to the normalized score by multiplying the normalized score by the defined importance weighting, resulting in weighted normalized scores for each dimension. The weighted normalized scores (WNS) are aggregated and multiplied by 100 to calculate the MDRI score. For example, an MDRI score of 88 points corresponds to high severity and requires immediate action, systemic improvements and organizational learning due to severe patient harm, systemic process failures, and high risk of recurrence.

Examples of systemic improvements include closed-loop communication systems, technology integration, and staff education. For example, closed-loop communication systems can be configured to ensure that all critical test results require acknowledgement and action confirmation from the responsible clinician. For example, technology integration can involve leveraging electronic medical record (EMR) tools to automate test result notifications with built-in escalation pathways and alerts for TPADs. For example, staff education can involve training staff on the importance of TPAD management, escalation protocols, and responsibilities for follow-up.

Examples of organizational learning include standardized policies and monitoring dashboards. For example, standardized policies can involve developing clear policies for managing TPADs, including defining critical test results and escalation pathways. For example, monitoring dashboards can involve using real-time dashboards to track pending results and follow-up actions to prevent delays in treatment for patients.

In this example, since the MDRI flagged the incident as a high-severity incident, necessitating immediate action and systemic improvements by implementing closed-loop communication systems, revising discharge protocols, and leveraging technology, the organization can mitigate the risk of future TPAD-related failures, improve patient safety, and ensure compliance with best practices.

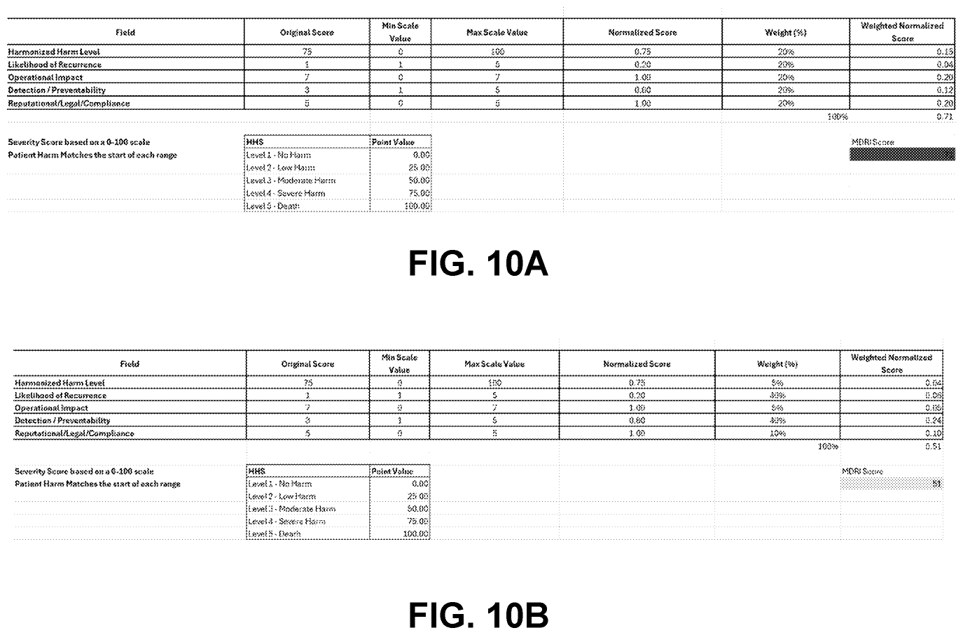

FIG. 11 shows an example user interface dashboard for users to score events or incidents on multiple dimensions of risk, according to some embodiments.

The user interface dashboard shown in FIG. 11 provides a mechanism for receiving input from users (e.g., risk managers) to score events on multiple dimensions of risk. The HHS is a fixed determined score based on the event, but all other dimensions of risk are configurable by users using the scales in FIG. 11. The manual scores result in a MDRI score, which shows overall risk to the organization (supplemental to patient harm severity). The scales shown in FIG. 11 represent configurable weights for the multiple dimensions of risk. Organizations can set the weights for each dimension during setup of the system, and these weights determine how much each dimension contributes to the final MDRI score and reflect organizational risk priorities.

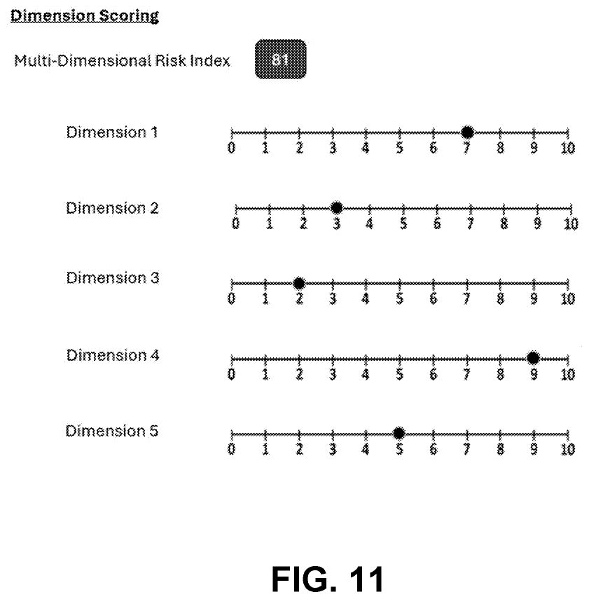

FIG. 12 shows an example user interface dashboard for users to submit details of events or incidents, according to some embodiments. Example details of events or incidents can include title, description, HHS score, and so on. The incident details entered into the user interface can be used by Risk Managers to assign contextual scores to each MDRI dimension. These manual inputs are important for accurate and context-sensitive risk evaluation.

In some embodiments, the assignment and validation of dimensional scores in the MDRI framework is managed centrally by the Risk Manager. This role functions as the system's coordinating authority, overseeing the consistent and contextually accurate application of risk assessment scoring. In scenarios where a reported incident includes contextual elements best understood by experts outside of traditional risk domains—such as, for example, Environmental Engineering, Facilities, or Outpatient Care—the Risk Manager is responsible for initiating a structured consultation with the appropriate departmental leader or subject matter expert.

Upon review of a new incident, the Risk Manager activates the MDRI scoring panel on a user interface. The Adjusted Harmonized Harm Score (HHS) is reviewed and validated or amended by the Risk Manager. In some embodiments, for any dimensional category (e.g., Operational Impact, Regulatory/Organizational Impact) that requires additional insight, the Risk Manager can select the corresponding specialty department via a collaboration function in the system. The system generates an automated alert to the designated leader or subject matter expert, inviting their input and review of the incident and its context. The specialty leader accesses the incident form, reviews the incident narrative, supporting materials, and HHS score, and enters proposed values for the dimension(s) under their purview. Once consensus is achieved, the Risk Manager finalizes the dimensional score(s), locks the MDRI panel, and submits the calculated MDRI score.

All collaboration entries, scoring adjustments, rationale, and user interactions are automatically documented within the audit trail, with time stamps and user credentials to ensure regulatory compliance and organizational accountability. This ensures that while the Risk Manager retains central responsibility and oversight of the MDRI process, the dimensional scores benefit from the domain expertise of interdisciplinary leaders. This results in more accurate, context sensitive scoring reflective of true risk potential across the care continuum to support both real-time and delayed response cycles, and ensuring that even high-complexity or specialty-related incidents are appropriately and proportionally weighted within the MDRI model.

In some embodiments, the processing system preserves, for each MDRI calculation, the configuration version that was active at the moment of computation. The persisted configuration version identifier captures the active dimension set, the per-dimension scoring scales, the per-dimension importance weights, the active missing/N/A handling rule, and any active mapping table from third-party harm scales to the HHS. When an event record is later viewed, audited, or compared against other events, the interface can display the configuration version under which the score was originally produced, and can re-render the historical score using the original configuration even after the organization's current configuration has changed. This per-calculation configuration provenance permits longitudinal analysis to distinguish genuine changes in underlying risk from changes attributable to model reconfiguration.

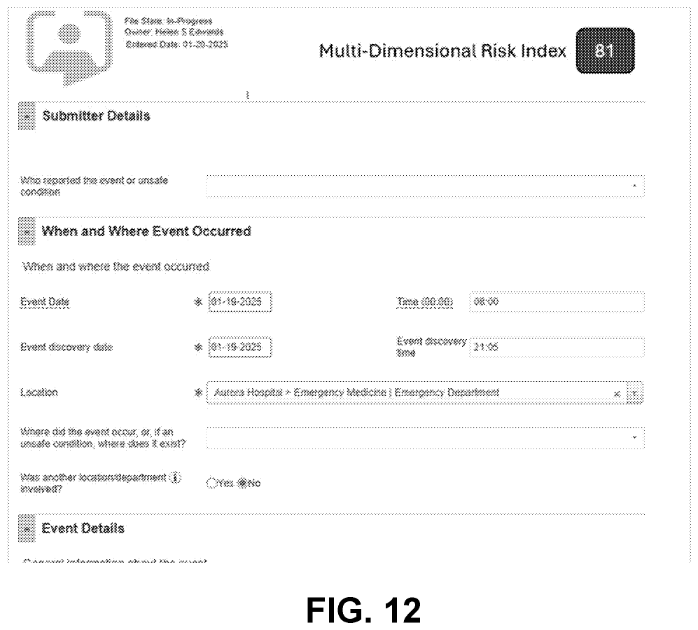

FIG. 13 shows a stacked bar chart visualization of example weighted normalized scores of multiple risk dimensions, according to some embodiments. FIG. 13 illustrates how the per-dimension weighted normalised scores that feed into the MDRI computation displayed side-by-side for individual incidents (or aggregated groups), with each bar segmented by dimension so the relative contribution of each dimension to the overall score is visually apparent.

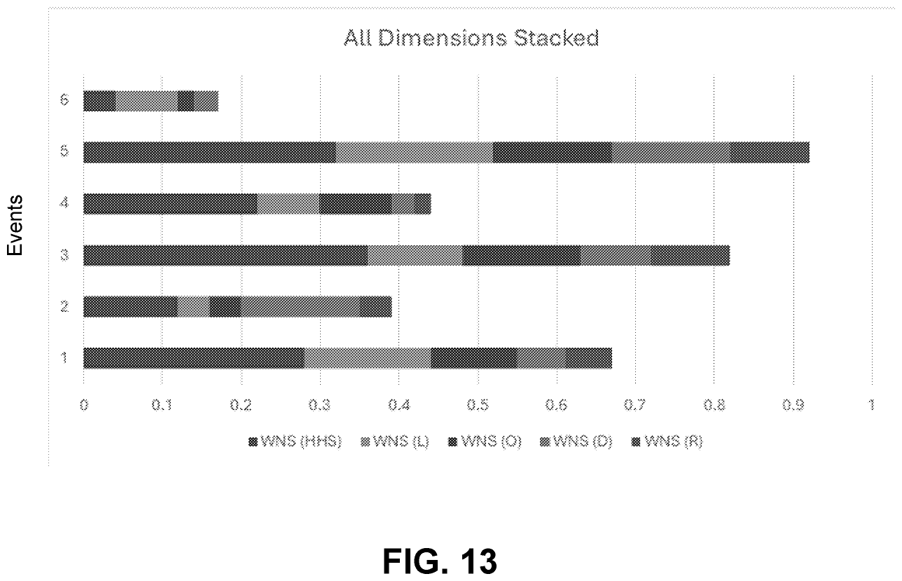

FIG. 14 shows a pie chart visualization of an example MDRI count of events, according to some embodiments. In the example as shown in FIG. 14, incidents or events with a MDRI score in the moderate range (31-60) take up the majority of events, followed by those in the high range (81-100), the low range (0-30), and lastly the high range (61-80).

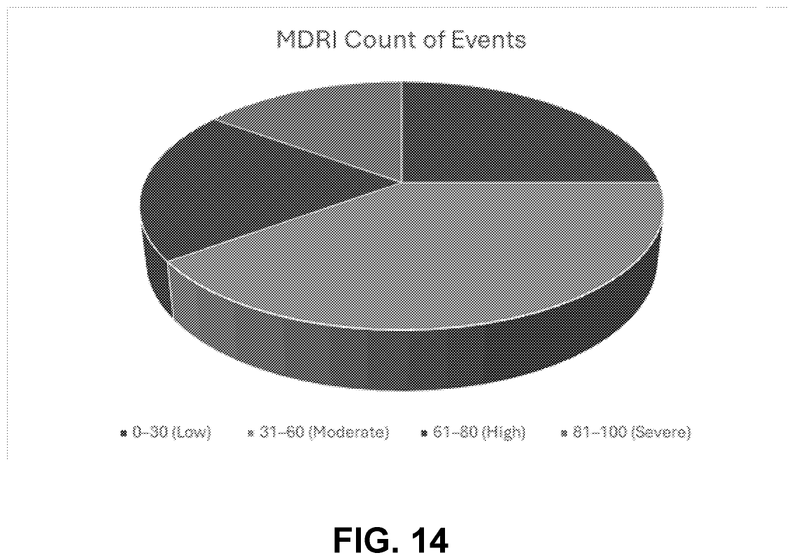

FIG. 15 shows a line graph visualization of an example average final MDRI score trend (across all events), according to some embodiments. In FIG. 15, the average final MDRI score trends higher as time passes through the year, starting at approximately 45 in January and growing to approximately 80 in December, illustrating time as one of many external factors exhibiting periodic influences on MDRI scores.

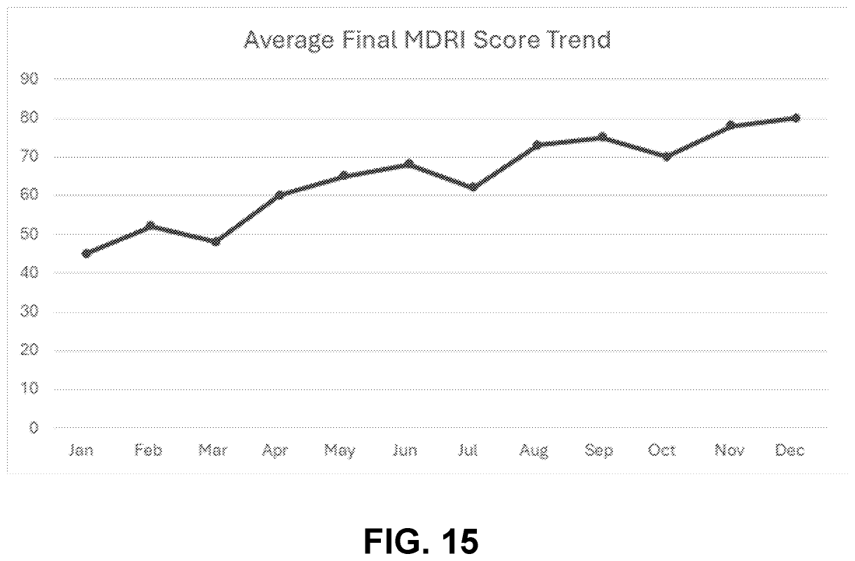

FIG. 16 shows an example Key Performance Indicator (KPI) style card, according to some embodiments. The KPI card shown in FIG. 16 displays an example of categorization with scores of at least 55 and above being categorized as significantly serious, and scores below 4 being categorized as little or no harm. In some embodiments, each dimension may be color coded at the MDRI level.

### Phase 3: Context-Based Risk Surveillance (CBRS)

Context-Based Risk Surveillance (CBRS) represents a continuous, systematic methodology leveraging the outputs of the MDRI to proactively manage patient safety risks in near real time within healthcare organizations. This framework not only offers forecasting capabilities and risk insights specific to a healthcare facility and its unique patient population but also integrates organizational priorities and resource allocation into the monitoring and response mechanisms.

CBRS utilizes established surveillance principles, adapted and modified from syndromic surveillance models, tailored specifically toward patient safety incidents and organizational risk events. The surveillance activities within CBRS consist of continuous, methodical collection, analysis, interpretation, and dissemination of MDRI data to effectively manage and mitigate patient harm proactively.

In some embodiments, the CBRS process can involves steps of data integration and collection, dynamic analysis, identification and interpretation of signals, interactive investigation tools, action-oriented reporting and interventions, and final incident reports and documentation.

Incident reports and corresponding MDRI scores (including Adjusted Harmonized Harm Scores and Dimensional scores) are continuously collected via standardized incident reporting systems. In some embodiments, data is captured daily and contextualized against patient-day or admission benchmarks, typically normalized per 1,000 patient-days, to maintain consistency with recognized healthcare reporting standards.

MDRI scores and incident frequencies are continuously assessed against a dynamically calculated baseline (Dynamic Average Rate). Control limits (Dynamic Upper Control limits, ±1 Sigma, ±2 Sigma) are recalculated daily, accounting for inherent variability and evolving patient safety risks.

In some embodiments, the dynamically calculated baseline (Dynamic Average Rate) used as the expected reference against which observed MDRI values and incident frequencies are compared is produced by a smoothing method applied to the organization's historical time series. In some embodiments, the smoothing method is a polynomial regression of degree n fitted to historical normalized MDRI values (or, equivalently, to normalized incident counts per 1,000 patient-days) over a configurable lookback window, expressed as: y=β0+β1·x+β2·x²+ . . . +βn·xⁿ+ε. In this expression, y is the expected risk value (the smoothed MDRI or smoothed incident rate) at a given reporting period, x is the time index (e.g., day, shift, or reporting period) measured from the start of the lookback window, β0 is the intercept, β1 through βn are the regression coefficients fitted from the organization's historical data, n is the polynomial degree (configurable per organization, with n=2 or n=3 being common defaults), and ¿ is the residual variation between fitted and observed values. The fitted polynomial defines the expected trajectory.

In some embodiments, the smoothing method is configurable, and polynomial regression is one of a plurality of selectable baseline-comparison methods. Other selectable methods include rolling averages over a configurable window, moving averages (including exponentially weighted moving averages), and statistical process control methods (including Shewhart control charts, CUSUM, and EWMA charts). The selected method is stored as part of the organization's CBRS configuration and is versioned together with the dimensional configuration so that the baseline-generation method active at the time of any given signal detection is preserved.

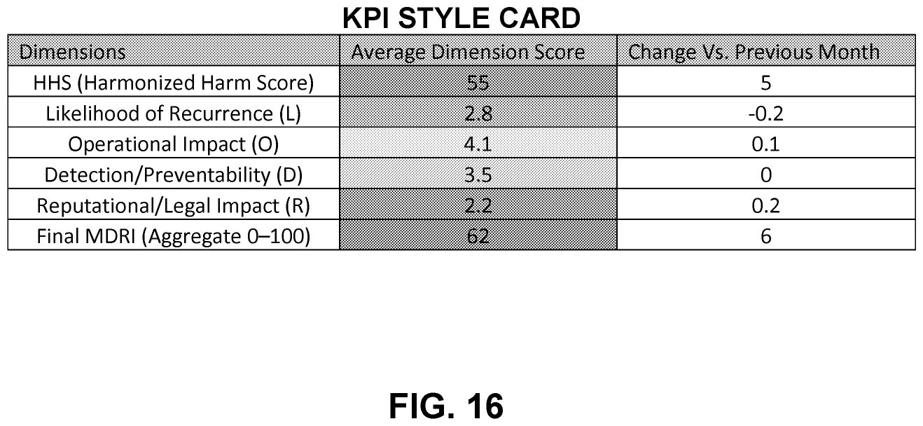

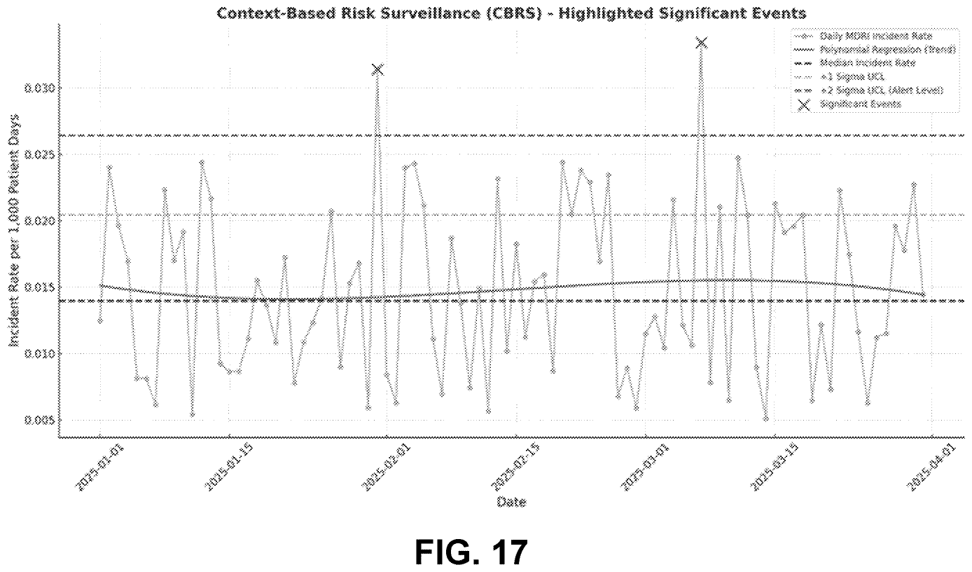

The fitted baseline produces an expected trajectory across the surveillance horizon. From this expected trajectory, the processing system derives, for each future reporting period in the horizon, an expected value y and a corresponding standard deviation σ (sigma) computed from the residuals ε of the fit over the lookback window. The processing system then constructs, for each reporting period, control bands at y±1σ and y±2σ. The expected trajectory and its control bands are recomputed on a configurable cadence (for example, daily) so that the bands evolve as the organization's underlying risk profile evolves and as new incidents enter the time series. The expected trajectory and its control bands are made available both to the visualization layer (for rendering in the line graphs of FIG. 17 and FIG. 18) and to the signal-detection layer described below.

As a worked example using a polynomial of degree n=2, suppose that for a given organization the fitted coefficients over a 30-day lookback window are β0=42, β1=0.85, and β2=0.03, with a residual standard deviation σ=6. At time index x=10 (day 10 of the surveillance horizon), the expected MDRI is y=42+(0.85)(10)+(0.03)(100)=42+8.5+3=53.5. The +1σ upper control limit at day 10 is therefore 53.5+6=59.5 (rounded to 60), and the +2σ upper control limit is 53.5+12=65.5 (rounded to 66). If the observed daily average MDRI on day 10 is 68, the observed value exceeds both the +1 sigma and +2 sigma thresholds, and the processing system records a +2σ exceedance for day 10 and triggers the corresponding signal-detection and intervention pathway.

In some embodiments, signal detection by the processing system proceeds as follows. For each reporting period, the processing system compares the observed value (per-1000-patient-day-normalized MDRI or normalized incident count) against the expected trajectory and the dynamically computed control bands. An observed value within ±1σ of the expected trajectory is recorded as nominal. An observed value exceeding +1σ but not +2σ is recorded as a watch-state signal and is annotated on the dashboard. An observed value exceeding +2σ is recorded as a sentinel signal, generates a Sentinel Event Alert, automatically opens an Investigator's Form pre-populated with the time window, the contributing incident identifiers, the active configuration version, and the expected vs. observed values, and dispatches an automated alert to the designated risk manager or investigation team via the routing rules of the active configuration. In some embodiments, the processing system also detects sustained sub-threshold drift (for example, k consecutive periods above the expected trajectory but below +1σ) using configurable run-rules, and surfaces these as drift signals distinct from threshold-crossing signals.

In some embodiments, intervention dispatch following signal detection is handled by an Intervention Engine of the processing system. On a sentinel signal, the Intervention Engine selects, from a configured library of interventions, one or more interventions whose triggering conditions match the signal context (for example, the dimensional pattern that drove the exceedance, the unit or care setting, the time of day, and the involved staff role). Each dispatched intervention is instantiated as a tracked work-item with a Grandparent>Parent>Child task hierarchy, assigned to a responsible role under the role-based access controls, and given a defined intervention window. Completion, in-progress, and not-applicable status updates flow back into the event record and into the next surveillance re-computation, so that the effect of the intervention on the subsequent observed trajectory can be measured and attributed. In some embodiments, the Intervention Engine returns, via the bidirectional API, the dispatched intervention payload to the calling external system so that downstream operational systems (for example, EMR task lists or staff messaging systems) can act on the same intervention without duplicate data entry.

Incidents crossing established control limits (e.g., exceeding +1 or +2 sigma thresholds) trigger notifications and automated alerts indicating a statistically significant deviation from historical trends. The system automatically flags such data points for follow up further detailed investigation by designated risk managers or incident investigation teams.

The system can use a variety of interactive investigation tools. Examples of interactive investigation tools include:

Sentinel Event Alert (Red X Icon): Indicates to users that the data point exceeds the ±2 sigma threshold and should be investigated for context. On clicking the symbol all cases making up that indicator will cascade and allow for selection of the individual incident form and take users directly to that form. Facilitating the initiation of the investigation for correlation, causation and context.

Investigator Notes (Red Teardrop Icon): Allows users/investigators real-time documentation of investigator insights, allowing attachment of supporting documents and visual evidence directly linked to specific incidents or risk events.

Intervention Window (Green Barbell Icon): Represents time-bound intervention periods, stretching from identification of risk to closure of the incident investigation or resolution of contributing factors.

Intervention Tracker (Pink Donut Icon): Provides hierarchical checklists for actionable interventions categorized by customizable grandparent, parent, and child logic. Users can document interventions completed or underway to mitigate risks identified.

CBRS supports healthcare leaders and risk managers by providing action-oriented reporting and interventions through clear visualization of emerging trends and risk hot spots via dashboards, such as: stacked dimension charts displaying MDRI dimensional breakdown, 3D pie charts representing the proportional distribution of MDRI risk events, trend line charts tracking average MDRI scores over defined periods, and KPI cards summarizing dimensional performance and change indicators month-over-month.

At the conclusion of the investigation and intervention processes of CBRS, the system automatically compiles a comprehensive report. This includes all relevant MDRI data, investigator notes, implemented interventions, attached documentation, and visual evidence. Reports provide a transparent, actionable narrative of each risk event, facilitating organizational learning, accountability, and proactive management of patient safety and organizational risk.

By integrating patient-specific and organizational-level risk data, CBRS offers healthcare leadership a perspective on organizational risk health, facilitating informed strategic decisions driven by context specific data. CBRS changes reactive risk management into proactive surveillance, enabling early identification and interventions before incidents escalate. CBRS systematically prioritizes patient safety interventions and resource allocation based on validated, real-time analytics, enhancing overall patient safety culture and organizational resilience.

The CBRS workflow involves continuous MDRI data collection and normalization, dynamic recalculation of control limits based on daily MDRI scores, automatic identification of significant deviations and initiation of alerts (e.g., those points outside of ±2 sigma), interactive investigation tools for documenting and tracking responses, visualization of trends for executive decision-making and strategic intervention planning and resource assignment, and automatically generated final reports for accountability, learning, and continuous quality improvement.

This CBRS framework complements and leverages HHS and MDRI systems, delivering strategic patient safety and risk surveillance capabilities within the healthcare facility.

CBRS supports healthcare leaders and risk managers by providing action-oriented reporting and interventions through clear visualization of emerging trends and risk hot spots via dashboards, such as: stacked dimension charts displaying MDRI dimensional breakdown, 3D pie charts representing the proportional distribution of MDRI risk events, trend line charts tracking average MDRI scores over defined periods, and KPI cards summarizing dimensional performance and change indicators month-over-month.

FIG. 17 shows a line graph visualization of incident rates obtained using Context-Based Risk Surveillance (CBRS) over time with highlighted significant events, according to some embodiments. The visualization in FIG. 17 shows a polynomial regression trend line that illustrates the underlying trend in daily incident rates, a median incident rate line that represents the central tendency to identify days above or below the typical daily incident rate, a +1 Sigma UCL line that highlights the upper threshold of standard variation, a +2 Sigma UCL line to indicate a critical alert threshold signalling significant anomalies or variance against the client's background rate. The visualization in FIG. 17 also shows significant events (represented as markers) which are highlighted clearly above the critical limit.

FIG. 18 shows a line graph visualization of average MDRI scores over time with highlighted significant scores, according to some embodiments. The visualization in FIG. 18 includes highlighted significant MDRI events (clearly indicated by markers) above the critical “+2 Sigma” alert level, the polynomial regression trendline demonstrating the data trajectory, a median MDRI score and Sigma thresholds (+1 Sigma UCL and +2 Sigma UCL) to facilitate clear interpretation of data. This visualization demonstrates significant MDRI scores, which comprise or consist of multiple events, for quick identification and management.

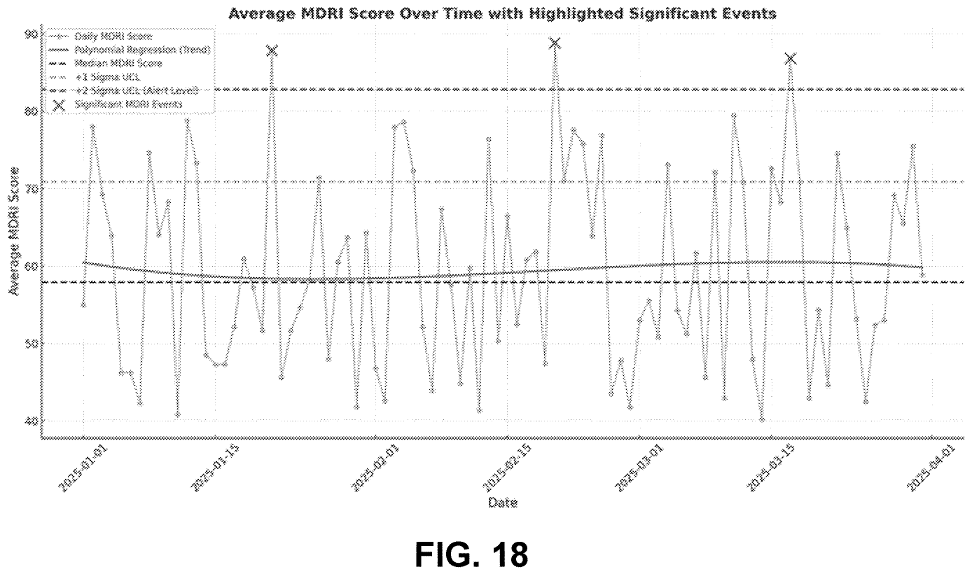

FIG. 19 shows a heat map visualization of MDRI scores per day over 31 days, utilizing a gradient color-coding system, according to some embodiments.

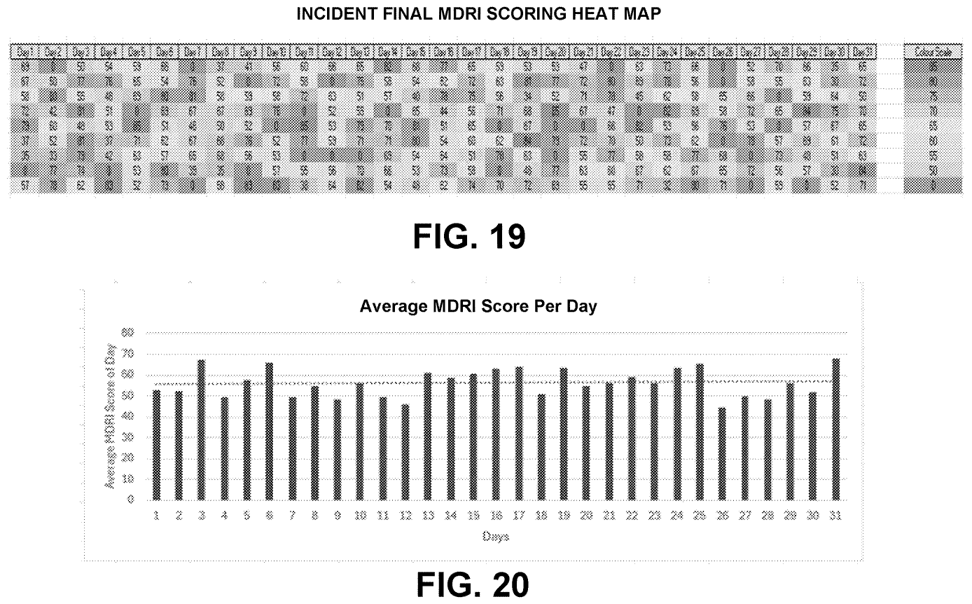

FIG. 20 shows a bar graph visualization of average MDRI scores per day over 31 days, according to some embodiments.

Visualizations help to clearly convey different insights from the MDRI data, such as trends, comparisons, intensity and distribution, and cumulative trends. In some embodiments, the visualization layer exposes a drill-through path from each dashboard element into the underlying records that produced it. From an MDRI value displayed on a KPI card (e.g., FIG. 16), a stacked dimension chart (e.g., FIG. 13), a heat map cell (e.g., FIG. 19), or a control-band line graph (e.g., FIGS. 17-18), the user may click through to a list of the individual incident records that contributed to the displayed aggregate, and from each incident record the user may navigate further into the dimensional breakdown, the configuration version used, the investigator notes, the dispatched interventions, and any AI-assisted scoring rationale. The drill-through path operates against the same RBAC-governed data store, so that a user navigating from the dashboard sees only those underlying incident records that the user is authorized to view.

### Phase 4: Investigation, Review, and Systemic Learning

CBRS is designed to detect, stratify, and contextualize healthcare risks using historical baselines, control charts, and contextual variables (e.g., time of day, staffing ratios, handoff points). In some embodiments, an Investigator's Form is used after a statistical or context-sensitive deviation is detected. Examples of such deviations include: a spike in wrong-patient medication errors during shift change, a rise in patient falls on night shifts, an outlier in procedure-related complications after a process change, and the like. This acts as a human-in-the-loop layer that transitions data signals into actionable clinical intelligence as the Investigator's Form functions as a contextual investigation and interpretation tool. Using such a tool enables investigators to capture environmental, staffing, and process factors that help distinguish random variation from actionable risk.

Tools such as the Investigator's Form further support the MDRI and drive systemic learning. The form captures qualitative judgments, such as preventability, potential severity, repeatability, and operational impact, which map directly into the MDRI model, enriching surveillance granularity. Findings can be categorized and looped back into policy updates, workflow redesign, and training focus as a learning system instead of just a reporting system.

In some embodiments, the Investigator's Form (or Investigator's Form) presented by the processing system includes a defined set of structured fields, including without limitation: an Investigator Name field; an Investigation Start Date field; a Date of Incident Review field; an investigator-confirmed HHS slider (which, when submitted, becomes the system HHS); a Contributing Factors checklist (with selectable categories such as communication breakdown, equipment, training, staffing, environment, and policy); a preventability indicator with three states (Yes/No/Uncertain); one or more file-upload fields for photographs, witness statements, and supporting documents; an electronic-signature capture field for the investigator and, where applicable, for a co-reviewing subject-matter expert; and a “Flagged for CBRS Dashboard” checkbox that, when selected, causes the incident to be persistently surfaced on the CBRS dashboard for follow-up regardless of whether the underlying MDRI has crossed a sigma threshold. Each structured field is timestamped and user-attributed, and changes to any field after initial submission are recorded as versioned amendments rather than overwrites.

In a practical exemplary scenario, CBRS flags a 30% increase in medication scanning overrides through event reports containing MDRI scoring. An investigator would open the Investigator's Form and note that new barcode scanners were installed last week with no formal training. Staff may report frequent scanner misreads and delays. The risk stratification of this scenario would be high preventability and moderate harm potential. The action would be to notify the pharmacy or IT for a device audit and initiate micro-training for staff.

In some embodiments, an intervention tracker is implemented as part of the system. The intervention tracker can be launched directly from the CBRS dashboard, linked to specific data points that exceed predefined thresholds (e.g., +2 Sigma alerts). The intervention tracker can be designed to accommodate Grandparent>Parent>Child category logic, the structure enables highly customizable intervention documentation. It supports both predefined options and the ability for users to add custom interventions based on unique risk profiles or evolving incident types. The intervention tracker would enable progressive documentation of interventions over time. Risk managers or leadership can mark interventions as Planned, In Progress, Completed, or Not Applicable. Entries from multiple roles can be supported (e.g., Risk Management, Clinical Leadership).

In some embodiments, the intervention tracker is integrated with role-based access controls (RBAC) to segment who can view, edit, or finalize interventions. In the dimensional scoring, a risk manager's collaborative role is the assignment and validation of dimensional scores within the Multi-Dimensional Risk Index (MDRI) framework which is fundamentally managed by the Risk Manager, who serves as the central oversight authority for risk assessment within the system. However, recognizing the multidisciplinary nature of incidents, the Risk Manager can actively engage and collaborate with equivalent leadership colleagues in specialized areas, such as Environmental Engineering, Outpatient Care, and other relevant disciplines. As an example, for an event with a low MDRI score but a high reputational risk, the Risk Manager can intervene granularly at the individual dimension level and set a high reputational score so that the system can initiate a notification to the reputational dimension owner to intervene further on the risk.

For incidents where specific expertise beyond the risk manager's immediate domain is required, the risk manager can consult directly with their corresponding departmental leaders or subject matter experts. Through this collaborative dialogue, appropriate dimensional weighting can be accurately determined, ensuring that each dimension's potential impact is comprehensively assessed and reflects the insights and priorities of each relevant discipline. Such collaboration ensures dimensional scores accurately represent organizational contexts and specialties, thereby promoting informed, proactive risk management interventions tailored to the unique aspects of each incident.

Every intervention checklist is tied to a specific incident ID and its MDRI score, enabling both qualitative and quantitative analysis of the impact of interventions on risk trends. Interventions can be exported in different formats, such as, for example, individual incident summaries, monthly/quarterly intervention effectiveness reports, and aggregated data for exporting to other systems. In some embodiments, the system provides customizable dashboards that allow for visualization of most commonly used interventions, completion rates, time-to-completion per intervention, and outcome metrics (linked to incident recurrence rates or MDRI reduction), and the like. Each user interaction is timestamped, with full edit history and digital signature capture if needed for regulatory or legal compliance. Completed intervention forms can also be flagged for meetings and reviews, such as, for example, Patient Safety Rounds, Monthly QI Meetings, Board-Level Reviews.

In some embodiments, the processing system implements a closed-loop feedback architecture in which inputs received through the Investigator's Form modify downstream computations and analytics. Specifically, an investigator's update to the system HHS or to any dimensional score causes the processing system to recompute the MDRI for the event under the re-computation triggers described above, to write a new MDRI record linked to the event, and to re-evaluate the event against the active CBRS control bands. The investigator's preventability indicator and contributing-factors checklist are persisted with the event and are surfaced on the dashboard as event annotations. The investigator's determination of whether the corresponding sentinel signal was actionable is captured as a structured “actionable signal” tag, which feeds a longitudinal analytics layer that measures, for each intervention type and each unit, the rate of recurrence of similar events and the change in the per-1,000-patient-day MDRI in the periods following intervention completion. This layer permits the processing system to report, over time, whether interventions are reducing recurrence and lowering measured risk, and to surface intervention types whose effect is statistically detectable versus those whose effect is not.

In a practical exemplary implementation, the system can be embedded directly into a source application of a risk-based incident reporting tool. Administrators of the reporting tool can build an HHS field into the reporting tool to capture events. From within the administrator area of the reporting tool application, the administrators can define MDRI-related settings through an administrator panel of the reporting tool application. The MDRI-related settings can be configured to identify the dimensions, capture other MDRI-related or CBRS-related information if not already captured by the reporting tool (e.g., number of beds, patient-days, etc.) and define conditions to trigger email alerts. The MDRI-related settings can be configured to identify the dimensions by defining dimension names, dimension owners and subject matter experts, define dimension scales and scoring systems, and define importance weightings for the dimensions. The reporting tool application can include a UI that can be configured to capture the dimensional risk scores, which can be defined directly in the reporting tool or could be provided through a widget or microservice. The reporting tool system can be configured to transmit the dimensional risk scores from the reporting tool to the system to calculate the MDRI score and store the MDRI score in the reporting tool application (e.g., on the incident form). The reporting tool application itself can then use its own internal alerting, notification, reporting, and dash-board tools to use the MDRI and dimensional risk data. In this scenario, the system is used as a calculation engine for the MDRI score and the reporting tool application handles the input of data and initiation of an action or intervention.

In another practical exemplary implementation, the system is configured to be a standalone tool. The system can be configured to be a standalone tool that organizations can access (outside of the event or incident reporting tool), allowing the organization to identify dimensions of risk, capture other MDRI-related or CBRS-related information if not already captured by the reporting tool (e.g., number of beds, patient-days, etc.), perform MDRI computations, and initiate actions or interventions such as email alerts, notifications, or the like. The system can be configured to identify the dimensions by defining dimension names, dimension owners and subject matter experts, define dimension scales and scoring systems, and define importance weightings for the dimensions. The system can be configured to have UI elements that can be exposed to other applications (e.g., as part of a RESTful or GraphQL API) so that they can embed form fields, inputs, or settings from the MDRI system into their application and/or event forms as necessary. If they choose to not embed UI elements into their event forms, organizations can also embed an MDRI UI interactive element (e.g., a button) into their event forms that redirects to a UI of the system itself to be managed independently (e.g., I/O interface 110). Users from organizations can then define dimension values using a tool either embedded in the reporting tool or residing in the MDRI system. The dimension values can be transmitted via an API (e.g., as an HTTPS request) to the MDRI system (e.g., in JSON or XML format). Once received, the system will compute the MDRI score and transmit the calculated values to the organizations' external application via the same API that the initial request was sent through (e.g., again, via a HTTPS response). The system can be further configured to have a separate internal service to handle alerting and notifications. The reporting tool application can then use the results to update its own UI or use them in further processing.

Each client or organization would need a tenant within the MDRI system to access where the MDRI settings and data reside. The MDRI settings can be stored in a table of the database (where the dimensions, scales for each dimension, and importance weighting are defined), and data values would be stored in another table or series of related tables (where data related to the event ID from the reporting tool application would be stored along with dimension scores provided and any other relevant information that should be captured).

### Computational Implementation.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Devices, systems, methods, and non-transitory computer-readable storage mediums configure healthcare risk management systems to integrate scoring systems including a Harmonized Harm Score (HHS), a Multi-Dimensional Risk Index (MDRI), and Context-Based Risk Surveillance (CBRS).

Devices, systems, methods, and non-transitory computer-readable storage mediums configure healthcare risk management systems for capturing and computing risk measurements for use with different healthcare systems.

Devices, systems, methods, and non-transitory computer-readable storage mediums configure healthcare risk management systems to integrate different data sources for multi-dimensional data risk analytics to proactively detect, prioritize, and mitigate risks in real-time to improve patient safety and operational resilience in healthcare systems.

Devices, systems, methods, and non-transitory computer-readable storage mediums configure healthcare risk management systems for integration and interoperability of the scoring systems (HHS, MDRI, CBRS), customizable weighting and normalization tools, and real-time analytics and evidenced and defined interventions enabled by the system.

Embodiments can be executed on distributed or cloud-based platforms to ensure scalability, flexibility, and interoperability across diverse healthcare environments.

Throughout the foregoing discussion, numerous references were made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

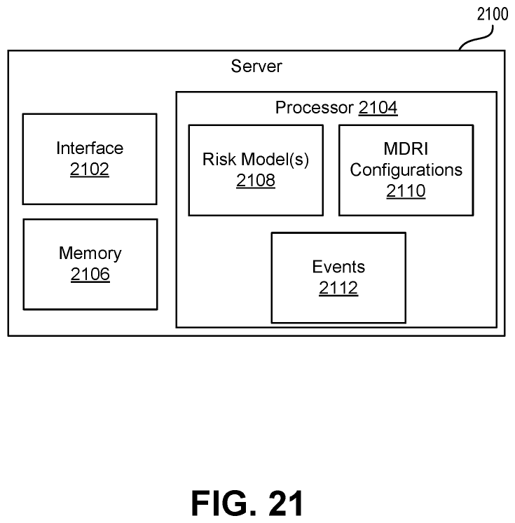

FIG. 21 shows an example schematic for a system for healthcare risk management according to some embodiments.

Server 2100 includes at least one processor 2104, memory 2106, at least one I/O interface 2102. The server 2100 components may be connected in various ways including directly coupled, indirectly coupled via a network, and distributed over a wide geographic area and connected via a network.

The non-transitory memory 2106 stores one or more risk models and different configurations for model dimensions and weights.

The processor 2104 is configured to receive the configurations for model dimensions and weights, and update the configurations stored in the memory, and receive input data from a plurality of input data streams, and standardize the input data. Upon detecting a healthcare event, processor 2104 computes a multidimensional risk index for the incident using a risk model of different dimensions, and the different configurations for model dimensions and weights. The processor 2104 is configured to initiate an action or event based on the multidimensional risk index;

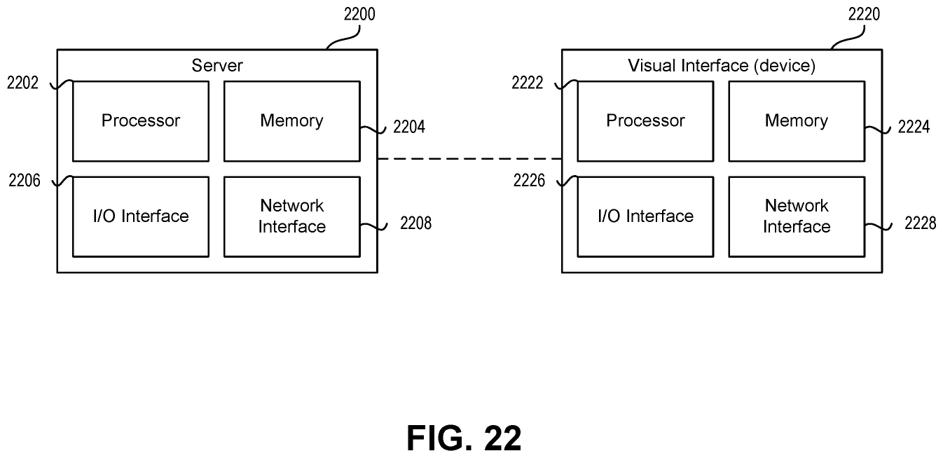

FIG. 22 shows an example schematic for a system for healthcare risk management with a visual interface according to some embodiments.

Server 2200 includes at least one processor 2202, memory 2204, at least one I/O interface 2206, and at least one network interface 2208. The server 2200 components may be connected in various ways including directly coupled, indirectly coupled via a network, and distributed over a wide geographic area and connected via a network. Visual interface 2220 resides on a device that includes at least one processor 2222, memory 2224, at least one I/O interface 2226, and at least one network interface 2228. The server 2200 and visual interface 2220 may be connected in various ways to exchange data. Visual interface 2220 can be a client application configured to indicate different dimensions for the model and receive the configurations for model dimensions and weights. Visual interface 2220 generate visual elements at least in part corresponding to a portion of the multidimensional risk index, and the event.

In some embodiments, system 100, server 2200, and visual interface 2220 are implemented as a special-purpose machine purpose-built for high-throughput, low-latency multidimensional risk computation, regression-based smoothing, and context-based risk surveillance, rather than as a general-purpose computer running ordinary software. In such embodiments, processor 2202 comprises one or more application-specific integrated circuits (ASICs), field-programmable gate arrays (FPGAs), tensor processing units (TPUs), graphics processing units (GPUs), or other hardware accelerators configured to execute, in parallel and at line rate, the standardisation, weighted-normalisation, and MDRI computation operations described herein. Memory 2204 may comprise high-bandwidth memory (HBM), non-volatile dual in-line memory modules (NVDIMMs), and hardware-encrypted storage modules that maintain per-tenant cryptographic isolation through dedicated key-management hardware (e.g., a hardware security module (HSM) or trusted platform module (TPM)). Network interface 2208 may comprise a smart network interface card (SmartNIC) or data processing unit (DPU) configured to terminate the bidirectional application programming interface, perform input validation (range, active-dimension, and weight-sum-to-100% gating) in hardware, and route ingested data streams from external systems. Such external systems may include electronic medical record systems, laboratory systems, scheduling systems, nurse call systems, medication-dispensing cabinets, and patient-monitoring equipment. External data is ingested directly into accelerator memory without traversing the host operating system. Visual interface 2220, when implemented as a special-purpose machine, may include a dedicated graphics pipeline configured to render the dashboards, drill-through paths, sentinel/teardrop/barbell/donut iconography, heat maps, and stacked dimensional bar charts described herein at interactive frame rates across large tenant populations.

In further embodiments, the regression-based smoothing operation that fits the expected risk trajectory and derives the ±1σ and ±2σ control bands is offloaded to a hardware accelerator (e.g., a GPU, TPU, or FPGA) configured to perform the underlying matrix operations as fixed-function or firmware-defined pipelines, such that the trajectory and bands for each tenant and each MDRI series can be recomputed within deterministic time bounds whenever a re-computation trigger occurs. The same accelerator, or a companion accelerator, may be configured to evaluate the alternative smoothing methods disclosed herein as selectable hardware kernels, and to perform the comparison-against-current-activity stratification (nominal/watch/sentinel/drift) by streaming each newly computed MDRI value through a hardware comparator against the precomputed band coefficients. In such embodiments, the Intervention Engine may be implemented as a hardware-scheduled work-queue that issues role-based assignments and prefilled Investigator's Form payloads to downstream devices through the SmartNIC/DPU, and the closed-loop write-back of investigator inputs and outcomes into the next MDRI recomputation may be implemented as a hardware-accelerated message bus with cryptographic provenance, such that the per-calculation configuration version, the trajectory parameters, the band crossings, and the “actionable signal” tagging are persisted as a tamper-evident record.

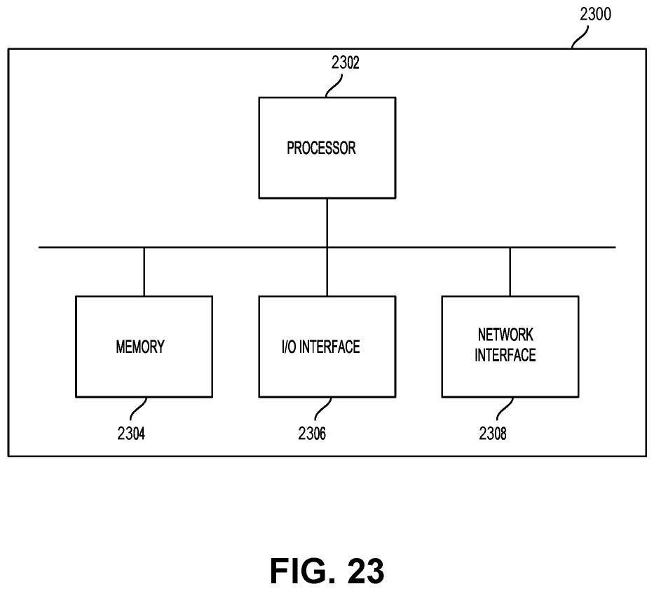

FIG. 23 is a schematic diagram of computing device 2300, according to some embodiments. As depicted, computing device 2300 includes at least one processor 2302, memory 2304, at least one I/O interface 2306, and at least one network interface 2308. The computing device components may be connected in various ways including directly coupled, indirectly coupled via a network, and distributed over a wide geographic area and connected via a network (which may be referred to as “cloud computing”).

For simplicity only one computing device 2300 is shown but the system may include more computing devices 2300 operable by users to access remote network resources and exchange data. The computing devices 2300 may be the same or different types of devices.

For example, and without limitation, the computing device 2300 may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

Each processor 2302 may be, for example, any type of microprocessor or microcontroller, a digital signal processing (DSP) processor, an integrated circuit, a field programmable gate array (FPGA), a reconfigurable processor, a programmable read-only memory (PROM), or any combination thereof.

Memory 2304 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 2306 enables computing device 2300 to interconnect with one or more input devices, such as a keyboard, mouse, camera, touch screen and a microphone, or with one or more output devices such as a display screen and a speaker.

Each network interface 2308 enables computing device 2300 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 2300 is operable to register and authenticate users (using a login, unique identifier, and password for example) prior to providing access to applications, a local network, network resources, other networks and network security devices. Computing devices 2300 may serve one user or multiple users.

### Practical Implementation Details.

Applicant notes that the described embodiments and examples are illustrative and non-limiting. Practical implementation of the features may incorporate a combination of some or all of the aspects, and features described herein should not be taken as indications of future or existing product plans. Applicant partakes in both foundational and applied research, and in some cases, the features described are developed on an exploratory basis.

The following discussion provides many example embodiments. Although each embodiment represents a single combination of inventive elements, other examples may include all possible combinations of the disclosed elements. Thus, if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only.

What is claimed is:

1. A healthcare risk management system, the system comprising:
a processing subsystem comprising one or more processors and one or more memories coupled with the one or more processors, the processing subsystem configured to cause the system to:
receive input data from a plurality of input data streams and standardize the input data;
receive or determine, for a detected healthcare event, harmonized harm scores at least in part of the standardized input data;
compute a current multidimensional risk index value for the healthcare event from the harmonized harm scores using a configurable risk model that defines a set of active dimensions, normalization values for each active dimension and weights for each active dimension, the current multidimensional risk index value comprising a total weighted normalized score across the active dimensions;
fit a smoothing model to a historical time series of multidimensional risk index values and derive an expected risk trajectory with dynamically computed control bands;
compare the current multidimensional risk index value against the expected risk trajectory and the dynamically computed control bands, and, upon an exceedance, initiate an action or intervention based on the multidimensional risk index value;
generate visual elements corresponding at least in part to a portion of the multidimensional risk index value and the action or intervention initiated; and
transmit the visual elements to an interface of a client device for display.

2. The system of claim 1, wherein the smoothing model is a regression-based model having the form y=β0+β1x+β2x²+ . . . +βnxⁿ+ε, where y is an expected multidimensional risk index value for a reporting period x, β0 is an intercept, β1 through βn are coefficients fitted from the time series of multidimensional risk index values, n is a polynomial degree, and ε is a residual variation.

3. The system of claim 1, wherein the smoothing model is selectable from a plurality of methods comprising a polynomial regression, a rolling average, a moving average, and an exponentially weighted moving average.

4. The system of claim 2, wherein the dynamically computed control bands are produced, from a standard deviation of the expected risk trajectory, at ±1 standard deviation and ±2 standard deviations from the expected risk trajectory.

5. The system of claim 1, wherein configurations for the configurable risk model indicate specific dimensions representing risk categories, normalization min scale values and max scale values for each dimension, and weights for each dimension.

6. The system of claim 5, further comprising the client device with the interface to receive updated configurations for the configurable risk model and display at least a portion of the visual elements generated at least in part corresponding to a portion of the multidimensional risk index, and wherein the processing subsystem is further configured to receive the updated configurations for the configurable risk model and recompute the current multidimensional risk index value for the healthcare event from the harmonized harm scores and generate updated visual elements for display at the interface.

7. The system of claim 1, wherein the processing subsystem is further configured to stratify a current multidimensional risk index value, by reference to the expected risk trajectory and the dynamically computed control bands, into one of: a nominal state, a watch state corresponding to a +1 standard deviation exceedance, a sentinel state corresponding to a +2 standard deviation exceedance, and a drift state corresponding to a sustained directional deviation from the expected risk trajectory over a configurable number of reporting periods, and select a structured intervention work-item in dependence upon the stratified state.

8. The system of claim 1, wherein the processing subsystem is further configured to ingest harmonized harm scores from, and route the action or intervention to, at least one of: a nurse call system, a medication-dispensing cabinet, a patient-monitoring device, and a scheduling system to initiate a downstream control action.

9. The system of claim 1, wherein the processing subsystem integrates scoring systems comprising one or more of: Harmonized Harm Score (HHS), a Multi-Dimensional Risk Index (MDRI), and Context-Based Risk Surveillance (CBRS).

10. The system of claim 1, wherein the processing subsystem further includes role-based access controls to provide access for risk model configuration, MDRI analysis, and intervention management to authorized users based on predefined access roles and organizational hierarchies.

11. A method for healthcare risk management, the method comprising:
receiving input data from a plurality of input data streams and standardizing the input data;
receiving or determining, for a detected healthcare event, harmonized harm scores at least in part from the standardized input data;
computing a current multidimensional risk index value for the healthcare event from the harmonized harm scores using a configurable risk model that defines a set of active dimensions, normalization values for each active dimension and weights for each active dimension, the current multidimensional risk index value comprising total weighted normalized score across active dimensions;
fitting a smoothing model to a historical time series of multidimensional risk index values and derive an expected risk trajectory with dynamically computed control bands;
comparing the current multidimensional risk index value against the expected risk trajectory and the dynamically computed control bands, and, upon an exceedance, initiating an action or intervention based on the multidimensional risk index value;
generating, for display at a visual interface, visual elements corresponding at least in part to a portion of the multidimensional risk index value and the action or intervention initiated; and
transmitting the visual elements to an interface of a client device.

12. The method of claim 11, wherein the smoothing model is a regression-based model having the form y=β0+β1x+β2x²+ . . . +βnxⁿ+ε, where y is an expected multidimensional risk index value for a reporting period x, β0 is an intercept, β1 through βn are coefficients fitted from the time series of multidimensional risk index values, n is a polynomial degree, and ε is a residual variation.

13. The method of claim 11, wherein the smoothing model is selectable from a plurality of methods comprising a polynomial regression, a rolling average, a moving average, and an exponentially weighted moving average.

14. The method of claim 12, wherein the dynamically computed control bands are produced, from a standard deviation of the expected risk trajectory, at +1 standard deviation and +2 standard deviations from the expected risk trajectory.

15. The method of claim 11, wherein configurations for the configurable risk model indicate specific dimensions representing risk categories, normalization min scale values and max scale values for each dimension, and weights for each dimension.

16. The method of claim 15, further comprising: receiving updated configurations for the configurable risk model and recomputing the current multidimensional risk index value for the healthcare event from the harmonized harm scores.

17. The method of claim 11, further comprising: stratifying a current multidimensional risk index value, by reference to the expected risk trajectory and the dynamically computed control bands, into one of: a nominal state, a watch state corresponding to a +1 standard deviation exceedance, a sentinel state corresponding to a +2 standard deviation exceedance, and a drift state corresponding to a sustained directional deviation from the expected risk trajectory over a configurable number of reporting periods, and selecting a structured intervention work-item in dependence upon the stratified state.

18. The method of claim 11, further comprising: ingesting harmonized harm scores from, and routing the action or intervention to, at least one of: a nurse call system, a medication-dispensing cabinet, a patient-monitoring device, and a scheduling system to initiate a downstream control action.

19. The method of claim 11, further comprising: controlling access for risk model configuration, MDRI analysis, and intervention management to authorized users based on predefined access roles and organizational hierarchies.

20. A non-transitory computer-readable medium to store instructions, which when executed by a computer, cause the computer to perform operations comprising:
receiving input data from a plurality of input data streams and standardizing the input data;
receiving or determining, for a detected healthcare event, harmonized harm scores at least in part based on the standardized input data;
computing a current multidimensional risk index value for the healthcare event from the harmonized harm scores using a configurable risk model, the current multidimensional risk index value comprising total weighted normalized score across active dimensions;
fitting a smoothing model to a historical time series of multidimensional risk index values and deriving an expected risk trajectory with dynamically computed control bands;
comparing the current multidimensional risk index value against the expected risk trajectory and the dynamically computed control bands, and, upon an exceedance, initiating an action or intervention based on the multidimensional risk index value;
generating, for display at a visual interface, visual elements corresponding at least in part to a portion of the multidimensional risk index value and the action or intervention initiated; and
transmitting the visual elements to an interface of a client device.
